(19) United States
(12) Patent Application Publication
(10) Pub. No.: US 2017/0166900 A1
(43) Pub. Date: Jun. 15, 2017

(54) PEPTIDE NUCLEIC ACID MONOMERS AND OLIGOMERS

(71) Applicant: Ugisense AG, Muenchen (DE)

(72) Inventors: Thomas Lindhorst, Innsbruck (AT); Birgit Werner, Garching (AT); Holger Bock, Innsbruck (AT)

(21) Appl. No.: 15/311,704

(22) PCT Filed: May 15, 2015
(86) PCT No.: PCT/EP2015/000998

§ 371 Date: Nov. 16, 2016

(30) Foreign Application Priority Data
May 16, 2014 (DE) 10 2014 007 158.8

(51) Int. Cl. C12N 15/113; C07F 9/40; C07F 9/6512

(52) U.S. Cl. CPC C12N 15/1138; C07F 9/6512; C07F 9/40; C12N 15/113; C12N 2320/33; C12N 2310/3181; C12N 2320/51

(57) ABSTRACT

The invention relates to new peptide nucleic acid monomers and peptide nucleic acid oligomers comprising a dialkylamine side chain substituted with phosphonic acid ester group(s) or phosphonic acid group(s), and to the uses thereof.

The invention relates to new peptide nucleic acid monomers and peptide nucleic acid oligomers comprising a dialkylamine side chain substituted with phosphonic acid ester group(s), to the preparation and the uses thereof.

Peptide nucleic acids (PNAs) are synthetic DNA/RNA analogues with an N-(2-aminoethyl)glycine structure—see general formula (PNA). In the general formula, NB denotes a nucleobase; n the number of PNA units (n=0-50); and F and G represent substituents.

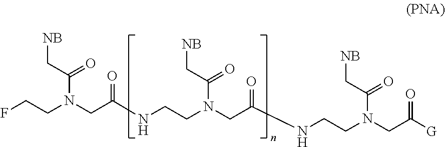

PNAs are prepared by creating peptide bonds between n-acetyl-N-(2-aminoethyl)glycine building blocks (PNA monomers). Each one of these individual N-acetyl-N-(2-aminoethyl)glycine building blocks represents a PNA unit.

The advantages of PNAs are that under physiological conditions they are resistant to hydrolytic (enzymatic) splitting, recognise complementary nucleic acid sequences (DNA or RNA) in a sequence-specific manner, and are able to bond with these with high affinity. PNAs are therefore considered attractive compounds for biotechnological and/or medical applications, such as for example diagnostics, or in antisense therapy.

For successful use as an active substance in antisense therapy sufficient bioavailability in the living organism is essential. The antisense active substance must be available in a sufficient quantity for a therapeutic effect at the target site, the target RNA or DNA. This means that the antisense active substance following administration must penetrate in sufficient quantities first (i) the tissue, then (ii) the tissue cells and finally (iii) within the cells the cell compartment as far as the target RNA or DNA, in order to achieve an antisense effect to a therapeutically significant extent.

PNAs, however, have the disadvantage that, compared with DNA, they are hardly soluble in water and have difficulty penetrating cell membranes. Accordingly, the use of PNAs as an active substance in antisense therapy in living organisms is very limited, as demonstrated for example by Beth M. et al., Antisense & Nucleic Acid Drug Development (2002) 12:65-70) based on investigations into the absorption or bioavailability of PNAs in various organs and tissues. In their investigations, Beth M. et al. found that PNAs following intraperitoneal administration in rats were excreted again within 24 hours 90% unchanged. Only 2.5% to 4.5% of the peptide nucleic acids were absorbed by the kidneys, and in all other organs the figure was actually significantly less than 1%.

To improve certain properties of PNAs such a water solubility, bonding properties to complementary DNA or RNA or absorption in cells, inter alia a modification of the PNAs through the introduction of a group R at the alpha-position of the PNA unit according to the following general formula (PNA*) has been proposed:

By way of example, as group R for modification of PNAs, side chains of natural amino acids have been proposed (Püschl A.

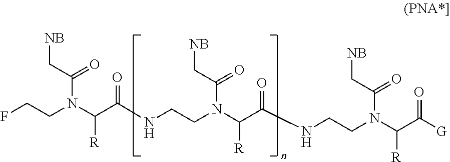

et al., Tetrahedron Letters 39, (1998) 4707-4710; U.S. Pat. No. 5,719,262). Although the steric hindrance is increased by the modification, degradation of the bonding of PNAs modified in this way to complementary DNA/RNA is only minimal, as established by measurements of the melting point of PNA/DNA hybrids (Püschl A. et al., loc. cit.).

PNAs with a lysine-modified basic structure (R═—(CH₂)₄—NH₂) demonstrated an improved water solubility and an increased melting point of PNA/DNA hybrids (U.S. Pat. No. 5,719,262). The disadvantage of these lysine modified PNAs, however, is that following absorption these remain trapped within the cell in the intracellular endosomes (Nielsen P., Quarterly Reviews of Biophysics 38, 4 (2005), 345-350; Koppelhus U. et al., Antisense & Nucleic Acid Drug Development (2002) 12:51-63). As a result, PNAs modified in this way are not sufficiently available within the cell for bonding to RNA and are thus unusable for a therapeutic application. As R. Corradini et al., Current Topics in Medicinal Chemistry, 11 (12), pp. 1535-1554, (2011) observed, such lysine modified PNAs, as for example described in U.S. Pat. No. 5,719,262, are not absorbed in some cells any way, and even if they were absorbed in the cell, they often remained trapped in vesicles. In the selection of cells investigated, no absorption in the cell nucleus was observed (loc. cit., p. 1543).

PNAs with an arginine modified basic structure, known as guanidine-based peptide nucleic acids (GPNAs):

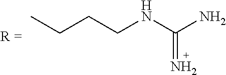

have been proposed for improving the absorption of PNAs in cells (Zhou P. et al., J. AM. CHEM. SOC. (2003) 125:6878-6879). Following cell absorption, GPNAs are localised intracellularly in the endoplasmic reticulum (ER) and can thus be available for bonding to the cell's own mRNA. However, in the living organism, following systemic administration, GPNAs are only absorbed by the kidneys, liver and tumour tissue (Thomas S. M. et al., ACS Chem. Biol. 2013 Feb. 15; 8(2):345-352). This limited bioavailability prevents a broader application of the GPNAs as therapeutic agents.

EP 1157031, EP 2041161 and Posch W. et al., Mol Med. (2012) 18: 111-22, disclose alkyl phosphonic acid ester modified PNAs with R═—(CH₂)ₙ—P═O(OEt)₂, (n=1,2). PNAs modified in this way are better able to enter cells and have better water solubility than PNAs not containing this modification. Furthermore, PNAs modified in this way are able, in HIV, to demonstrate an effectiveness across two generations of virus.

Compared with GPNAs, these alkyl phosphonic acid ester group modified PNAs also have better bioavailability. Alkyl phosphonic acid ester group modified PNAs have the disadvantage, however, that their water solubility is dependent upon the nucleobase sequence. Thus, for example, in a sequence containing a large number of guanine and cytosine bases, the water solubility of these PNAs is reduced. This sequence-dependent water solubility can make therapeutic use more difficult.

To allow PNAs to be used widely as a therapeutic agent they must combine a number of different properties: good, sequence-independent water solubility; good cell absorption; good bonding properties to DNA and/or RNA; good bioavailability (the better the bioavailability in various tissues, the more possibilities for therapeutic use are available); long half-lives (in order to achieve bonding of the PNA to the DNA and/or RNA also in a cell in the living organism and to obtain the desired effect there of modulation of the gene expression); good bonding to blood plasma proteins to support bioavailability and extend the half-life (oligomers bonded to blood plasma proteins are not filtered out from the bloodstream so quickly in the kidneys and excreted via the urine); and a powerful effect of the modulation of the gene expression, for which good cell absorption and intracellular distribution and good bonding properties of the PNA to DNA and/or RNA are also necessary.

The disadvantage of the known modified PNAs is that they only have some of the important properties of (i) good and sequence-independent water solubility, (ii) good cell absorption and intracellular distribution, (iii) good bioavailability and long half-lives in as many therapeutically relevant tissues as possible, (iv) good bonding to blood plasma proteins and/or (v) a powerful effect of the modulation of the gene expression, but do not have all the properties necessary for broad application as a therapeutic agent.

The object of the invention is thus to provide new modified PNA monomers; and to provide new modified PNA oligomers with improved property profiles, containing the new PNA monomers as building blocks. The improved properties profile relates to the combination of the properties of (i) good and sequence-independent water solubility, (ii) good cell absorption and intracellular distribution, (iii) good bioavailability and long half-lives in as many therapeutically relevant tissues as possible, (iv) strong bonding to blood plasma proteins and (v) powerful effect of the modulation of the gene expression.

A further object of the invention is to provide new methods for application of the abovementioned modified PNA oligomers, and diagnostic and therapeutic compositions containing said modified PNA oligomers.

This and other aspects of the invention will become clear from a consideration of the following detailed description and definitions.

The invention relates to:

[1] a compound of general formula (I):

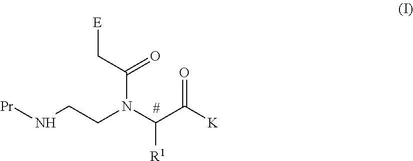

wherein

K represents a carboxylic acid active ester group or —O—R_{M}; wherein R_{M} represents an H atom, a methyl group, ethyl group, allyl group, benzyl group, phenyl group, tert-butyl group, or a trimethylsilyl group;

Pr represents an H atom or an amino protective group;

# denotes an asymmetric C atom;

E is an H atom, a phenyl group, a heterocycle, a nucleobase, or a nucleobase substituted with a nucleobase protective group;

R¹ is a group, represented by the general formula (II):

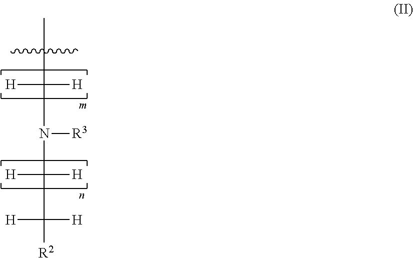

wherein

R² is a phosphonic acid ester group or a phosphonic acid group;

R³ is an H atom, or an amino protective group;

m represents an integer from 1 to 5; and

h represents an integer from 0 to 4;

provided that for the sum of m and h in the general formula (II) 2≦x≦5.

Bonding the R¹ group to the backbone of the monomeric compound according to the general formula (I) results in an asymmetric centre (#) in the backbone at the point of bonding of R¹ and backbone. At each such asymmetric centre in the backbone (#) there can be either an R-configuration or an S-configuration. Here the configuration at this asymmetric centre (#) is defined along the lines of the Cahn-Ingold-Prelog sequence rules, with the further condition that the priority of the ligands is always defined as follows: the nitrogen atom at the asymmetric centre is always given priority 1. The carbon atom of the carboxyl group at the asymmetric centre is always given priority 2. The carbon atom of group R¹ at the asymmetric centre is always given priority 3. The hydrogen atom at the asymmetric centre is always given priority 4.

The term carboxylic acid active ester group designates the carboxylic acid derivatives known to a person skilled in the art, which are normally used in peptide chemistry to increase the coupling reactivity of the carboxylic acid function. Such carboxylic acid active ester groups are, for example, described in: O. Marder, F. Albericio, Chimica Oggi, 2002, 37; N. Sewald, H.-D. Jakubke, (eds), Peptide Chemistry, Wiley-VCH Verlag, Weinheim 2002, Chapter 4.3 Peptide Bond Formation, Page 197. Examples of a carboxylic acid active ester group are carboxylic acid halides, acyl phosphonium salts such as tris(pyrrolidino)-phosphonium carboxylate (by reaction with PyBroP), anhydrides, thiophenyl esters, cyanomethyl esters, nitro esters and dinitrophenyl esters, pentafluorophenyl esters, chlorophenyl esters, trichlorophenyl esters, pentachlorophenyl esters, and the active esters listed in the following table.

| | | | (Short) name of the |
|---|---|---|---|
| | Generally | | reagent with which the |
| | used | | activating group, inter |
| Activating group | symbol | Structure | alia, is introduced |
| N-hydroxypiperidinyl | OPip | 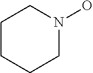 | N-nydroxypiperidine (HOPip) |
| 8-quinolyl | OQ | 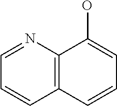 [SMILES: Oc1cccc2cccnc12] | 8-Hydroxyquinoline |
| N-hydroxysuccinimidyl | OSu | 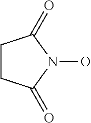 | N-Hydroxysuccinimide HOSu N,N′- disuccinimidyl carbonate |
| 1-hydroxybenzotrizolyl | OBt | 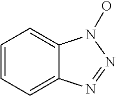 | HOBt, BOP, PyBOP HBTU, TBTU |
| 7-aza-1-hydroxybenzo- triazolyl | OAt | 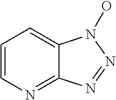 | HOAt, PyAOP, HATU, HAPyu, HAPipU, HAMDU, HAMTU |
| 6-chloro-1-hydroxy- benzotriazolyl | ClOBt | 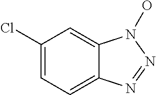 [SMILES: On1nnc2ccc(Cl)cc21] | HCTU |
| N-norbornen-2,3- dicarboximidooxy | ONdc | 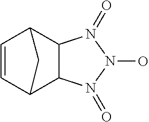 | n-Hydroxy-5-norbornen-2,3- dicarboximide HONB or HONdc |
| Ethyl-1-hydroxy-1H- 1,2,3-triazol-4- carboxylat | OCt | 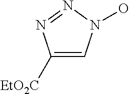 [SMILES: CCOC(=O)c1cn(O)nn1] | Ethyl-1-hydroxy-1H-1,2,3- triazol-4-carboxylate HOCt |

The term amino protective group designates protective groups known to a person skilled in the art, used in the organic synthesis of amino acids or peptides, for example a trifluoracetyl, oxocarbamate, thiocarbamate, fluorenylmethoxycarbonyl (Fmoc), carbobenzoxy (Cbz), monomethoxytrityl (Mmt), phthaloyl, t-butoxycarbonyl (Boc), benzhydryloxycarbonyl (Bhoc), or an allyloxycarbonyl (Alloc) protective group.

The term nucleobases, designates bases known to a person skilled in the art capable of base pairing with DNA bases or RNA bases. Examples of nucleobases include bases with a purine basic structure, for example adenine, guanine, hypoxanthine, xanthine and 7-methylguanine; or a pyrimidine basic structure, for example cytosine, uracil, thymine, 5-hydroxymethylcytosine, 5-methylcytosine, and 5,6-dihydrouracil; as well as analogues and bioisosteres thereof.

The term nucleobase protective group designates protective groups known to a person skilled in the art, used in the organic synthesis of compounds with nucleobases, for example an acetyl, isobutyryl, benzyloxycarbonyl, diphenylmethyl, benzhydryloxycarbonyl, anisoyl, 4-tert-butylbenzoyl, benzyl or diphenylcarbamoyl group.

The term alkyl refers to a saturated, linear or branched hydrocarbon group, having 1 to 40 carbon atoms, preferably 1 to 20 carbon atoms, more preferably 1 to 12 carbon atoms, and particularly preferably 1 to 6 carbon atoms, for example, the methyl, ethyl, propyl, isopropyl, n-butyl, isobutyl, tert-butyl, n-pentyl, n-hexyl, 2,2-dimethylbutyl or n-octyl group.

The terms alkenyl and alkinyl refer to at least partially unsaturated, linear or branched hydrocarbon groups, having 2 to 40 carbon atoms, preferably 2 to 20 carbon atoms, more preferably 2 to 12 carbon atoms, and particularly preferably 2 to 6 carbon atoms, for example the ethenyl, allyl, acetylenyl, propargyl, isoprenyl or hex-2-enyl group. Alkenyl groups preferably have one or two (particularly preferably one) double bonds or alkenyl groups have one or two (particularly preferably one) triple bonds.

The term aryl or Ar refers to an aromatic group, having one or more rings, and 6 to 14 ring carbon atoms, preferably 6 to 10 (in particular 6) ring carbon atoms. Concrete examples are benzene, naphthalene or biphenyl.

The term aralkyl refers to groups which according to the above definitions contain both aryl and alkyl, alkenyl, alkinyl and/or cycloalkyl groups, such as, for example, arylalkyl, arylalkenyl, arylalkinyl, arylcycloalkyl, arylcycloalkenyl, alkylarylcycloalkyl and alkylarylcycloalkenyl groups. Concrete examples of aralkyls are toluene, xylene, mesitylene, styrene, 1H-indene, tetraline, dihydronaphthalene, indanone, phenylcyclopentyl, cyclohexylphenyl, fluorene and indane. An aralkyl group preferably contains one or two aromatic ring systems (1 or 2 rings) with 6 to 10 carbon atoms and one or two alkyl, alkenyl and/or alkinyl groups with 1 or 2 to 6 carbon atoms and/or one cycloalkyl group with 5 or 6 ring carbon atoms.

The term cycloalkyl refers to a saturated or partially unsaturated (e.g. cycloalkenyl) cyclic group, having one or more rings (preferably 1 or 2), and containing 3 to 14 ring carbon atoms, preferably 3 to 10 (in particular 3, 4, 5, 6 or 7) ring carbon atoms. Concrete examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, Tetralin, cyclopentylcyclohexyl, or a cyclohex-2-enyl group.

The term alkylcycloalkyl refers to groups which according to the above definitions contain both cycloalkyl and alkyl, alkenyl or alkinyl groups, e.g. alkylcycloalkyl, cycloalkylalkyl, alkylcycloalkenyl, alkenylcycloalkyl and alkinylcycloalkyl groups. An alkylcycloalkyl group preferably contains a cycloalkyl group, having one or two rings, and 3 to 14 ring carbon atoms, preferably 3 to 10, in particular 3, 4, 5, 6 or 7, ring carbon atoms; and one, two or three, preferably 1 or 2, alkyl, alkenyl or alkinyl group(s) each with 1 or 2 to 6 carbon atoms; wherein a C₄-C₁₁ alkylcycloalkyl group is preferred, and a C₄-C₇ alkylcycloalkyl group is particularly preferred. Concrete examples of alkylcycloalkyl groups are a methylcyclopropyl (C₄), methylcyclobutyl (C₅), ethylcyclopropyl (C₅), methylcyclopentyl (C₆), propylcyclopropyl (C₆), ethylcyclopentyl (C₇), methylcyclohexyl (C₇), ethylcyclopentenyl (C₇), or an ethylcyclohexadienyl (C₈) group.

The terms alkyloxy, alkenyloxy, alkinyloxy, alkyloxyaryl, and cycloalkyloxy refer to an alkyl, alkenyl, alkinyl, alkylaryl or cycloalkyl group, as indicated above, containing one or more —O groups. Examples are a methoxy, ethoxy, furan, tetrahydrofuran, or a 4-methoxybenzyl group.

The term heterocycle refers to a cycloalkyl group, an aryl group or an aralkyl group, as indicated above, in which one or more, preferably 1, 2 or 3, carbon atoms are replaced by an oxygen, nitrogen or sulphur atom. Examples are the piperidyl, piperazinyl, morpholinyl, urotropinyl, pyrrolidinyl, tetrahydrothiophenyl, tetrahydropyranyl, tetrahydrofuryl or 2-Pyrazolinyl group. The term heterocycle also covers, by way of example, an aromatic group, having one or more rings, and containing 5 to 14 ring atoms, preferably 5 to 10, in particular 5 or 6 ring atoms, wherein one or more, preferably 1, 2, 3 or 4, are oxygen, nitrogen, or sulphur ring atoms. Examples are the 4-pyridyl, 2-imidazolyl, 3-phenylpyrrolyl, triazolyl, oxazolyl, triazolyl, tetrazolyl, isoxazolyl, indazolyl, indolyl, benzimidazolyl, pyridazinyl, chinolinyl, purinyl, carbazolyl, acridinyl, pyrimidyl, 2,3′-bifuryl, 3-pyrazolyl and isochinolinyl groups.

The term amino acid refers to a carboxylic acid, in which one or more hydrogen atoms on a carbon atom are replaced by an amino group. An amino acid can by way of example be an α-amino acid such as glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine, histidine, selenocysteine, pyrrolysine, thyroxine, DOPA and L-ornithine, 5-hydroxytryptophane, lanthionine, β-chloroalanine, 2-methylalanine, citrulline, canavanine, theanine, cucurbitin, an β-amino acid such as β-alanine, or a γ-amino acid such as γ-aminobutyric acid (GABA).

The invention further comprises:

[2] The compound described in [1], wherein K represents —O—R_{M} and R_{M} is as defined in [1].

[3] The compound described in [1] or [2], wherein R_{M} represents an H atom, a methyl, an ethyl, an allyl or a trimethylsilyl group.

[4] The compound described in [1] to [3], wherein Pr represents an amino protective group.

[5] The compound described in [4], wherein the amino protective group is selected from an oxocarbamate, a thiocarbamate or an Mmt protective group.

[6] The compound described in [4] or [5], wherein the amino protective group is a Fmoc, Boc, Cbz, Bhoc, Alloc or an Mmt protective group.

[7] The compound described in [4] to [6], wherein the amino protective group is a Boc or a Fmoc protective group.

[8] The compound described in [1] to [7], wherein E represents an adeninyl, cytosinyl, pseudoisocytosinyl, guaninyl, thyminyl, uracilyl or phenyl group substituted as necessary with a nucleobase protective group.

[9] The compound described in [8], wherein E represents a group, selected from:

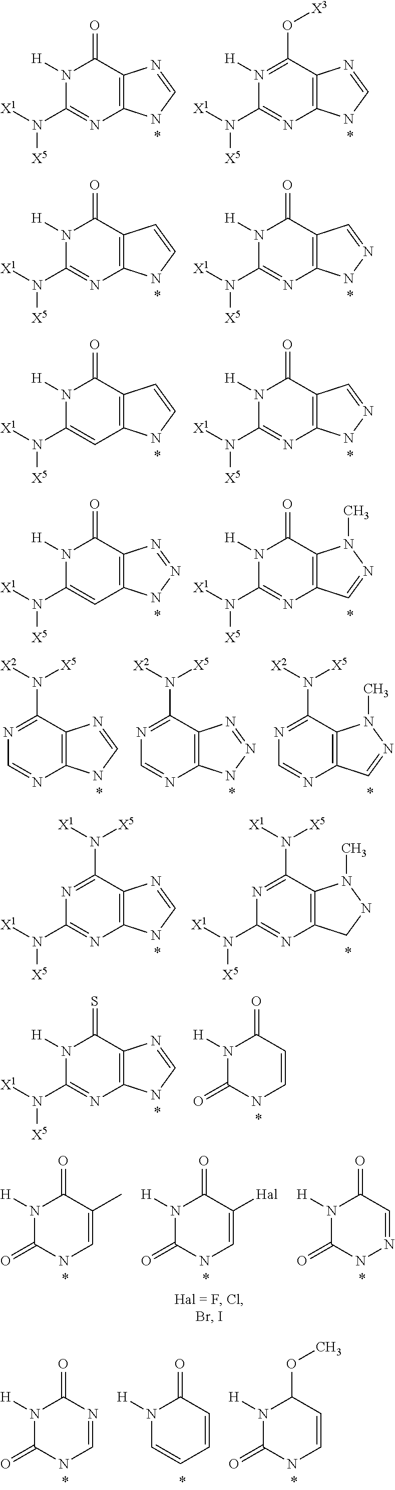

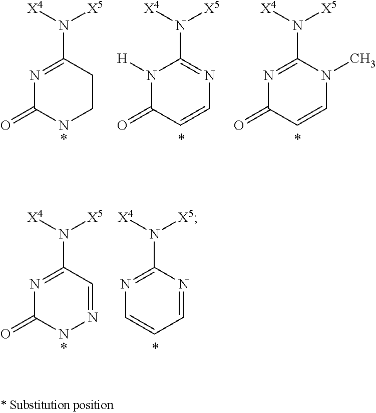

wherein

X¹-X⁴ in each case independently represent an H atom or a nucleobase protective group; and X⁵ in each case independently represents an H atom, or a Boc or Bhoc protective group.

[10] The compound described in [9], wherein X¹, X² and X⁴ in each case independently represent an H atom, acetyl (Ac), tert-butyloxycarbonyl (Boc) isobutyryl (iBu-CO), benzyloxycarbonyl (Cbz), (4-methoxyphenyl)-diphenylmethyl (Mmt), benzhydryloxycarbonyl (Bhoc), anisoyl (An), or 4-tert-butylbenzoyl (tBuBz);

X⁵ in each case independently represents an H atom, or a Boc or Bhoc protective group; and

X³ in each case independently represents an H atom, benzyl (Bn), or diphenylcarbamoyl (Dpc).

[11] The compound described in [1] to [10], wherein E represents a thyminyl group, uracilyl group, phenyl group, N2-acetyl-guaninyl group, N2-isobutyryl-guaninyl group, N2-benzyloxycarbonyl-guaninyl group, N2-(4-methoxyphenyl)-diphenylmethyl-guaninyl group, N2-benzhydryloxycarbonyl-guaninyl group, N2-di-benzhydryloxycarbonyl-guaninyl group, N2-tert-butyloxycarbonyl-guaninyl group, N2-di-tert-butyloxycarbonyl-guaninyl group, N6-enzyloxycarbonyl-adeninyl group, N6-(4-methoxyphenyl)-diphenylmethyl-adeninyl group, N6-anisoyl-adeninyl group, N6-benzhydryloxycarbonyl-adeninyl group, N6-di-benzhydryloxycarbonyl-adeninyl group, N6-tert-butyloxycarbonyl-adeninyl group, N6-di-tert-butyloxycarbonyl-adeninyl group, O6-benzylguaninyl group, N2-acetyl-O6-diphenylcarbamoyl-guaninyl group, N2-isobutyryl-O6-diphenylcarbamoyl-guaninyl group, N2-benzyloxycarbonyl-O6-diphenylcarbamoyl-guaninyl group, N2-(4-methoxyphenyl)-diphenylmethyl-O6-diphenylcarbamoyl-guaninyl group, N2-benzhydryloxycarbonyl-O6-diphenylcarbamoyl-guaninyl group, N4-benzyloxycarbonyl-cytosinyl group, N4-(4-methoxyphenyl)-diphenyl-methyl-cytosinyl group, N4-4-tert.butylbenzoyl-cytosinyl group, N4-benz-hydryloxycarbonyl-cytosinyl group, N4-di-benzhydryloxycarbonyl-cytosinyl group, N4-tert-butyloxycarbonyl-cytosinyl group, N4-di-tert-butyloxycarbonyl-cytosinyl group, N2-benzyloxycarbonyl-pseudo-isocytosinyl group, N2-(4-methoxyphenyl)-diphenylmethyl-pseudoisocyto-sinyl group, N2-4-tert.-butylbenzoyl-pseudoisocytosinyl group, N2-benz-hydryloxycarbonyl-pseudoisocytosinyl group, N2-di-benzhydryloxycarbonyl-pseudoisocytosinyl group, N2-tert-butyloxycarbonyl-pseudoisocytosinyl group or an N2-di-tert-butyloxycarbonyl-pseudoisocytosinyl group.

[12] The compound described in [1] to [11], wherein E represents a thyminyl group, uracilyl group, phenyl group, N2-benzyloxycarbonyl-guaninyl group, N2-benzhydryloxycarbonyl-guaninyl group, N2-tert-butyloxycarbonyl-guaninyl group, N2-benzyloxycarbonyl-O6-diphenylcarbamoyl-guaninyl group, N2-benzhydryloxycarbonyl-O6-diphenylcarbamoyl-guaninyl group, N6-benzyloxycarbonyl-adeninyl group, N6-benzhydryloxycarbonyl-adeninyl group, N6-tert-butyloxycarbonyl-adeninyl group, N6-di-tert-butyloxycarbonyl-adeninyl group, N4-benzyloxycarbonyl-cytosinyl group, N4-benz-hydryloxycarbonyl-cytosinyl group, N4-di-tert-butyloxycarbonyl-cytosinyl group, N2-benzyloxycarbonyl-pseudo-isocytosinyl group, N2-benz-hydryloxycarbonyl-pseudoisocytosinyl group or an N2-tert-butyloxycarbonyl-pseudoisocytosinyl group.

[13] The compound described in [1] to [12], wherein R² represents a phosphonic acid ester group of the formula —P(═O)(OV)₂ or —P(═O)(OV)(OH); and each V independently represents an unsubstituted C₁-C₇ alkyl, C₃-C₇ cycloalkyl, C₄-C₇ alkylcycloalkyl, phenyl, or benzyl group.

[14] The compound described in [13], wherein each V independently represents a methyl, ethyl, cyclohexyl, or benzyl group.

[15] The compound described in [14], wherein V in each case represents an ethyl group.

[16] The compound described in [1] to [15], wherein R³ is an H atom.

[17] The compound described in [1] to [15], wherein R³ represents an oxocarbamate, thiocarbamate, or an Mmt protective group.

[18] The compound described in [17], wherein R³ represents a Cbz, Alloc, Bhoc or Boc protective group.

[19] The compound described in [1] to [18], wherein m is 1, 2, 3 or 4.

[20] The compound described in [1] to [19], wherein h is 0, 1, 2, or 3.

[21] The compound described in [1] to [20], wherein R¹ represents a group of the formula —CH₂—CH₂—CH₂—CH₂—NH—CH₂—CH₂—P═O (OEt)₂, or a group of the formula —CH₂—CH₂—CH₂—NH—CH₂—CH₂—P═O (OEt)₂.

[22] The compound described in [1] to [15], [19] or [20], wherein R¹ represents a group of the formula —CH₂—CH₂—CH₂—CH₂—NR³—CH₂—CH₂—P═O (OEt)₂, or a group of the formula —CH₂—CH₂—CH₂—NR³—CH₂—CH₂—P═O (OEt)₂; and R³ is as defined in [17] or [18].

Compounds according to the invention of the general formula (I), as for example described in [1] to [22] above, can be used for preparing new oligomeric compounds. Accordingly, the invention further relates to:

[23] a compound comprising at least one repeat unit of the general formula (III):

—[Y_{d}—Z_{f}—Y_{g}—Zⱼ]ₙ— (III)

wherein

each Y in each case independently represents a group of the general formula (IV):

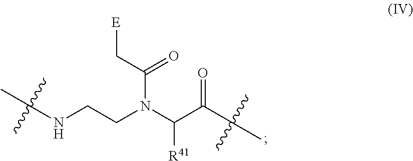

each Z in each case independently represents a group of the general formula (V):

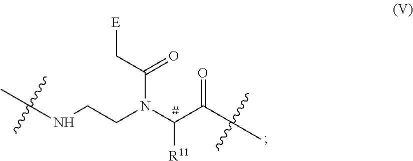

wherein

each E in each case independently represents an H atom, a phenyl group, a heterocycle, or a nucleobase;

# denotes an asymmetric C atom;

each R⁴¹ in each case independently represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine;

each R¹¹ represents a group —(CH₂)ₘ—NH—(CH₂)ₕ—CH₂—R¹²; wherein

R¹² in each case is a phosphonic acid ester group or a phosphonic acid group; m represents an integer from 1 to 5; and h represents an integer from 0 to 4; provided that for the sum of m and h: 2≦x≦5;

d in each case represents an integer from 0 to 5;

f in each case represents an integer from 0 to 5;

g in each case represents an integer from 0 to 5;

j in each case represents an integer from 0 to 5;

n in each case represents an integer from 1 to 10;

provided that the sum of all repeat units Y_{d}, Z_{f}, Y_{g}, and Zⱼ in the general formula (III) is ≦40 and at least one of the variables f or j an integer from 1 to 5.

If a repeat unit, e.g. formula (III), a group, e.g. Z or Y, or a substituent or a variable, e.g. E, R¹¹ or R⁴¹, appears more than once in a formula contained herein, each repeat unit, each group in the repeat unit, and each substituent or each variable is selected independently of each other, whether expressly indicated or not. By way of example, in formula (III) each group Z and Y and each variable E, R¹¹ or R⁴¹, respectively, is selected independently of each other. In other words, the general formula (III) contains at least one monomer unit Z according to the invention, as above, or by way of example defined in [1] to [22], and in total a maximum of 40 units Z or Z and Y.

By way of example, in the general formula (III) each group Y and Z, and each variable d, f, g and j is independently selected. Accordingly, the combination [Y_{d}—Z_{f}—Y_{g}—Zⱼ]ₙ with d, f, g, and j=1; and n=10 by way of example represents the following combination of units Y and Z: —[Y—Z—Y—Z—Y—Z—Y—Z—Y—Z—Y—Z—Y—Z—Y—Z—Y—Z—Y—Z—Y—Z—Y—Z—Y—Z—Y—Z—Y—Z—Y—Z—Y—Z—Y—Z—Y—Z—Y—Z]—, wherein the sum of the number of all units Y and Z is equal to 40. The combination [Y_{d}—Z_{f}—Yg—Zj], with d=3, f=1, g=1, and j=3; n=3 by way of example represents the following combination of units Y and Z: —[Y—Y—Y—Z—Y—Z—Z—Z—Y—Y—Y—Z—Y—Z—Z—Z—Y—Y—Y—Z—Y—Z—Z—Z]—, wherein the sum of the number of all units Y and Z is equal to 24.

It is also possible, however, for the variables d, f, g, and j in the respective repeat units [Y_{d}—Z_{f}—Y_{g}—Zⱼ] to differ from each other. By way of example, the following 4 repeat units [Y_{d}—Z_{f}—Y_{g}—Zⱼ]: (Y₁—Z₁—Y₁—Z₁), (Y₁—Z₁—Y₀—Z₀), (Y_{S}—Z₁—Y₀—Z₀), (Y₁—Z₁—Y₁—Z₁), i.e. n=4, could be combined in the following combination of groups Y and Z: —[Y—Z—Y—Z—Y—Z—Y—Y—Y—Y—Y—Z—Y—Z—Y—Z]—; the sum of the number of all groups Y and Z is equal to 16. Likewise, by way of example the following 3 (i.e. n=3) repeat units [Y_{d}—Z_{f}—Y_{g}—Zⱼ]: (Y₅—Z₁—Y₁—Z₁), (Y₁—Z₁—Y₀—Z₀), and (Y₁—Z₁—Y₅—Z₀) could be combined as follows: —[Y—Y—Y—Y—Y—Z—Y—Z—Y—Z—Y—Z—Y—Y—Y—Y—Y]—; the sum of the number of all groups Y and Z is equal to 17. Here, each group contained in the repeat units, or each substituent or each variable contained more than once in the repeat units according to the general formula (III), is in each case independently selected from the above definitions, whether expressly indicated or not.

The invention further comprises:

[24] a compound, represented by the general formula (VI):

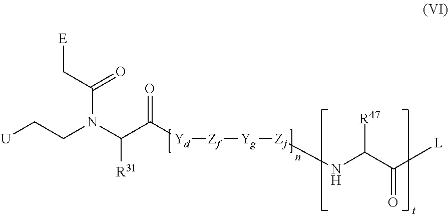

wherein

E, Y, Z, d, f, g, j and n in each case are independent as defined in [23]; provided that the sum of all repeat units Y_{d}, Z_{f}, Y_{g}, and Zⱼ in the general formula (VI) is ≦40 and at least one of the variables f or j represents an integer from 1 to 5;

R³¹ represents an H atom; a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; or a group —(CH₂)ₘ—NH—(CF₂)ₕ—CH₂—R¹²; wherein R¹² is a phosphonic acid ester group, or a phosphonic acid group; m represents an integer from 1 to 5; and h represents an integer from 0 to 4; provided that for the sum of m and h: 2≦x≦5;

R⁴⁷ in each case independently represents an H atom; a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; a group of the formula (IXb):

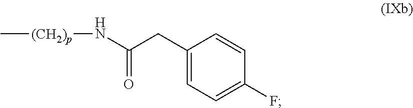

a group of the formula (IXc):

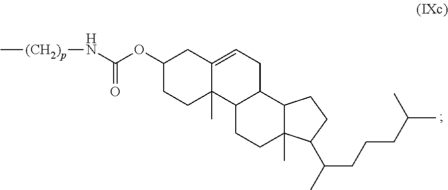

a group of the formula (IXd):

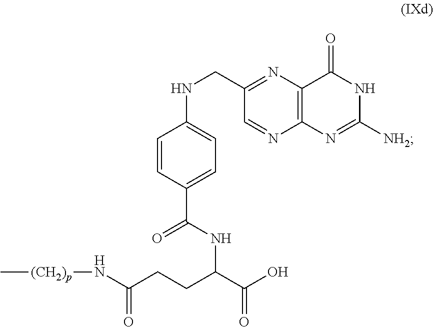

or a group of the formula (IXe):

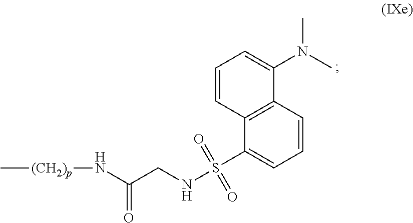

p in the formulae (IXb), (IXc), (IXd), and (IXe) represents the number 3 or 4;

t represents an integer from 0 to 10;

L represents —NR^{D}R^{E}—NHNR^{D}R^{E} or —OR^{F}; wherein R^{D}, R^{E} and R^{F} in each case independently of each other represent an H atom; or an alkyl, alkenyl, alkinyl, aryl, aralkyl, cycloalkyl, or cycloalkenyl group with in each case up to 20 C atoms;

U represents —NR^{A}R^{B}; —N^{⊕}R^{A}R^{B}R^{C}; —NR^{A}(CO)R^{B}; —NH(CO)NHR^{B}; —NH(CO)OR^{B}; a group of the formula (VIIIa):

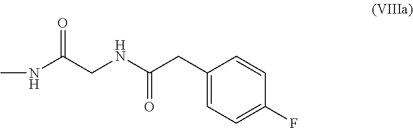 [SMILES: CNC(=O)CNC(=O)Cc1ccc(F)cc1]

a group of the formula (VIIIb):

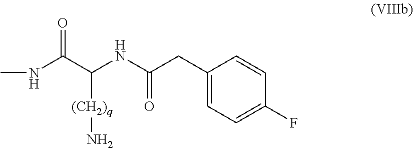

a group of the formula (VIIIc):

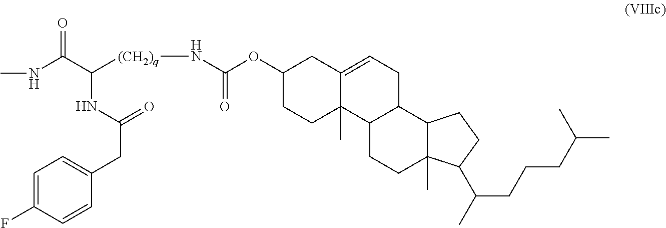

a group of the formula (VIIId):

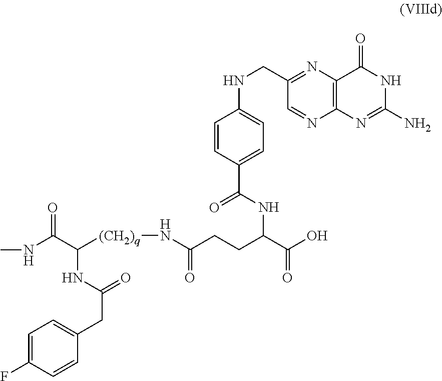

or a group of the formula (VIIIe):

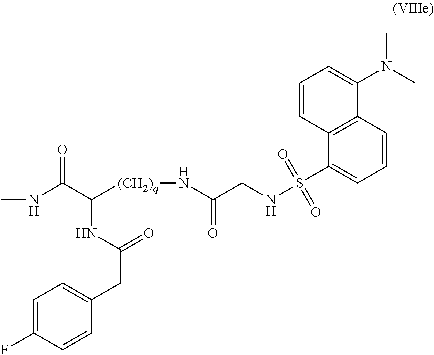

q in formulae (VIIIb), (VIIIc), (VIIId), and (VIIIe) represents the number 3 or 4;

or a group of the general formula (VII):

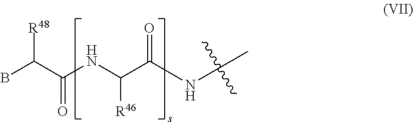

wherein

B represents an H atom, —NR^{H}R^{I}, —N^{⊕}R^{H}R^{I}R^{J}, —NR^{H}(CO)R^{I}—NH(CO)NHR^{I}, —NH(CO)OR^{I}, a phenyl group, or a substituted phenyl group, substituted with 1 to 3 substituents, selected from the group comprising OH, F, Cl, Br, I and NO₂;

each R^{A}, R^{c}, R^{H} and R^{J} in each case independently of each other represents an H atom, a methyl group or an amino protective group;

each R^{B} and R^{I} in each case independently represents an H atom; an amino protective group; an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group, with in each case up to 40 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group, one or more hydrogen atom(s) in each case independently of each other can be replaced by a phosphonic acid ester group or phosphonic acid group, F, Cl, Br, I, —OH, O—CH₃, S—CH₃, NO₂, ═O, NH₂, —S(O₂)NH—, —NHCH₃, —N(CH₃)₂, a C₁-C₆ alkyl, C₂-C₆ alkenyl, C₂-C₆ alkinyl, C₃-C₁₀ cycloalkyl, C₆-C₁₀ aryl or a C₇-C₁₂ aralkyl group;

R⁴⁸ and each R⁴⁶ in each case independently of each other represent an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine, or a group of the formula (IXb), (IXc), (IXd) or (IXe); and

s is an integer from 0 to 10.

[25] The compound described in [23] or [24], provided that the sum of all repeat units Y_{d}, Z_{f}, Y_{g}, and Zⱼ in the general formula (III) or (VI) is ≦30.

[26] The compound described in [23] to [25], provided that for the sum of all repeat units Y_{d}, Z_{f}, Y_{g}, and Zⱼ in the general formula (III) or (VI): 7≦x≦30.

[27] The compound described in [23] to [26], provided that for the ratio (sum of repeat units Z_{f} and Zj): (sum of all repeat units Y_{d}, Z_{f}, Y_{g}, and Zⱼ) in the general formula (III) or (VI): 0.1≦x≦1.0.

[28] The compound described in [23] to [26], provided that for the ratio (sum of repeat units Z_{f} and Zⱼ): (sum of all repeat units Y_{d}, Z_{f}, Y_{g}, and Zⱼ) in the general formula (III) or (VI): 0.1≦x≦0.8.

[29] The compound described in [23] to [26], provided that for ratio (sum of repeat units Z_{f} and Zⱼ): (sum of all repeat units Y_{d}, Z_{f}, Y_{g}, and Zⱼ) in the general formula (III) or (VI): 0.1≦x≦0.6.

[30] The compound described in [23] to [26], provided that for the ratio (sum of repeat units Z_{f} and Zⱼ): (sum of all repeat units Y_{d}, Z_{f}, Y_{g}, and Zⱼ) in the general formula (III) or (VI): 0.1≦x≦0.5.

[31] The compound described in [23] to [26], provided that for the ratio (sum of repeat units Z_{f} and Zⱼ): (sum of all repeat units Y_{d}, Z_{f}, Y_{g}, and Zⱼ) in the general formula (III) or (VI): 0.1≦x≦0.4.

[32] The compound described in [23] to [31], wherein each E in each case independently represents an adeninyl, cytosinyl, pseudoisocytosinyl, guaninyl, thyminyl, uracilyl, or phenyl group.

[33] The compound described in [23] to [32], wherein each R⁴¹ in each case independently represents an H atom, or a side chain of the amino acid lysine, ornithine, arginine, histidine, tryptophan, tyrosine, threonine or serine.

[34] The compound described in [23] to [33], wherein each R⁴¹ in each case independently represents an H atom, or a side chain of the amino acid lysine, ornithine, or arginine.

[35] The compound described in [23] to [34], wherein each R⁴¹ in each case represents an H atom.

[36] The compound described in [23] to [34], wherein each R¹² in each case independently represents a phosphonic acid ester group of the formula —P(═O)(OV)₂ or —P(═O)(OV)(OH); and each V in each case independently represents an unsubstituted C₁-C₇ alkyl, C₃-C₇ cycloalkyl, C₄-C₇ alkylcycloalkyl, phenyl, or benzyl group.

[37] The compound described in [36], wherein each V in each case independently represents a methyl, ethyl, cyclohexyl, or benzyl group.

[38] The compound described in [36] or [37], wherein V in each case represents an ethyl group.

[39] The compound described in [23] to [38], wherein each m in each case independently is 1, 2, 3 or 4.

[40] The compound described in [23] to [39], wherein each h in each case independently is 0, 1, 2, or 3.

[41] The compound described in [23] to [40], wherein each R¹¹ in each case represents a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

[42] The compound described in [23] to [41], wherein each d in each case independently is 0, 1, 2, 3 or 4.

[43] The compound described in [23] to [42], wherein each f in each case independently is 0, 1, 2, 3 or 4.

[44] The compound described in [23] to [43], wherein each g in each case independently is 0, 1, 2, 3 or 4.

[45] The compound described in [23] to [44], wherein each j in each case independently is 0, 1, 2, 3 or 4.

[46] The compound described in [23] to [45], wherein n=0, 1, 2, 3, 4, 5, 6, 7 or 8.

[47] The compound described in [24] to [46], wherein R³¹ represents an H atom, a side chain of the amino acid lysine, ornithine, arginine, histidine, tryptophan, tyrosine, threonine or serine, a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

[48] The compound described in [24] to [47], wherein R³¹ represents a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

[49] The compound described in [24] to [47], wherein R³¹ represents an H atom, or a side chain of the amino acid lysine, ornithine, or arginine.

[50] The compound described in [24] to [47] or [49], wherein R³¹ represents an H atom.

[51] The compound described in [24] to [50], wherein R⁴⁷ in each case independently represents an H atom; a side chain of the amino acid lysine, ornithine, arginine, histidine, tryptophan, tyrosine, threonine or serine; or a group of the formula (IXb), (IXc), (IXd) or (IXe).

[52] The compound described in [24] to [51], wherein R⁴⁷ in each case independently represents an H atom; or a side chain of the amino acid lysine, ornithine, or arginine.

[53] The compound described in [52], wherein R⁴⁷ in each case represents an H atom.

[54] The compound described in [52], wherein R⁴⁷ in each case independently represents a side chain of the amino acid lysine, ornithine, or arginine.

[55] The compound described in [24] to [54], wherein t=0, 1, 2, 3, 4, 5, 6, 7 or 8.

[56] The compound described in [24] to [51], wherein R⁴⁷ in each case independently represents an H atom; or a group of the formula (IXb), (IXc), (IXd) or (IXe); and t=1, 2, 3, or 4.

[57] The compound described in [56], wherein R⁴⁷ in each case independently represents a group of the formula (IXb), (IXc), (IXd) or (IXe).

[58] The compound described in [24] to [57], wherein L represents —OH, —NH₂, —NHNH₂, an —O(C₁-C₁₀) alkyl, —O(C₂-C₁₀)alkenyl, —O(C₂-C₁₀) alkinyl, —O(C₃-C₁₀) cycloalkyl, —O(C₄-C₁₁) alkylcycloalkyl, —O(C₆-C₁₀) aryl, —O(C₇-C₁₂) aralkyl, —NH—(C₁-C₁₀) alkyl, —NH(C₂-C₁₀) alkenyl, NH(C₂-C₁₀) cycloalkenyl, —NH(C₃-C₁₀)cycloalkyl, —NH(C₆-C₁₀) aryl, or an —NH(C₇-C₁₂) aralkyl group.

[59] The compound described in [24] to [58], wherein L represents —OH, —OEt, —NH₂ or —NHNH₂.

[60] The compound described in [24] to [59], wherein U represents —NR^{A}R^{B}; —NR^{A}(CO)R^{B}; —NH(CO)NHR^{B}; or —NH(CO)OR^{B}; R^{A} in each case represents an H atom or a methyl group; and R^{B} is as defined in [24].

[61] The compound described in [60], wherein R^{B} in each case represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 30 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester group or phosphonic acid group, F, Cl, Br, I, —OH, or NO₂.

[62] The compound described in [60], wherein R^{B} in each case represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 20 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester, or phosphonic acid, group, F, Cl, Br, I, —OH, or NO₂.

[63] The compound described in [60], wherein R^{B} in each case represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 12 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester group or phosphonic acid group, F, Cl, Br, I, —OH, or NO₂.

[64] The compound described in [24] to [59], wherein U represents a group of the general formula (VII); B—NR^{H}R^{I}, —NR^{H}(CO)R^{I}, —NH(CO)NHR^{I}, or —NH(CO)OR^{I}; R⁴⁸ is an H atom; each R⁴⁶ in each case independently of each other represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; and R^{H} and R^{I} are as defined in [24].

[65] The compound described in [24] to [59], wherein U represents a group of the general formula (VII); B—NR^{H}R^{I}, —NR^{H}(CO)R^{I}, —NH(CO)NHR^{I}, or —NH(CO)OR^{I}; R⁴⁸ represents a group of the formula (IXb) to (IXe); each R⁴⁶ in each case independently of each other represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; and R^{H} and R^{I} are as defined in [24].

[66] The compound described in [64] or [65], wherein R^{H} represents an H atom or a methyl group.

[67] The compound described in [64] to [66], wherein R^{I} represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 30 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester group or phosphonic acid group, F, Cl, Br, I, —OH, or NO₂.

[68] The compound described in [64] to [66], wherein R^{I} represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 20 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester group or phosphonic acid group, F, CI, Br, I, —OH, or NO₂.

[69] The compound described in [64] to [66], wherein R^{I} represents an H atom, an alkyl, alkenyl, alkinyl, aryl, aralkyl, cycloalkyl, alkylcycloalkyl, cycloalkenyl, alkyloxy, alkenyloxy, alkinyloxy, alkyloxyaryl, or a cycloalkyloxy group with in each case up to 12 C atoms; wherein in the alkyl, alkenyl, alkinyl, aryl, aralkyl, cycloalkyl, alkylcycloalkyl, cycloalkenyl, alkyloxy, alkenyloxy, alkinyloxy, alkyloxyaryl, or cycloalkyloxy group one or more hydrogen atom(s) in each case independently of each other can be replaced by a phosphonic acid ester group or phosphonic acid group, F, CI, Br, I, —OH, or NO₂.

[70] The compound described in [24] to [59], wherein U represents a group of the general formula (VII); B represents an H atom, a phenyl group, or a substituted phenyl group, substituted with 1 to 3 substituents, selected from the group comprising OH, F, Cl, Br, I and NO₂; R⁴⁸ is an H atom; and each R⁴⁶ in each case independently of each other represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine.

[71] The compound described in [64] to [70], wherein each R⁴⁶ in each case independently of each other represents an H atom, or a side chain of the amino acid alanine, arginine, asparagine, glutamine, histidine, isoleucine, leucine, lysine, methionine, ornithine, phenylalanine, proline, histidine, serine, threonine, tryptophan, tyrosine, or valine.

[72] The compound described in [64] to [70], wherein each R⁴⁶ in each case independently of each other represents an H atom, or a side chain of the amino acid arginine, histidine, lysine, methionine, ornithine, histidine, serine, threonine, tryptophan or tyrosine.

[73] The compound described in [64] to [72], wherein s=0, 1, 2, 3, 4, 5, 6, 7 or 8.

[74] The compound described in [24] to [59], wherein U represents a group of the general formula (VII); B represents an H atom, a phenyl group, or a substituted phenyl group, substituted with 1 to 3 substituents, selected from the group comprising OH, F, Cl, Br, I and NO₂; R⁴⁸ is an H atom; each R⁴⁶ in each case independently of each other represents an H atom, or a group of the formula (IXb) to (IXe); and s=1, 2, 3 or 4.

[75] The compound described in [74], wherein R⁴⁶ in each case independently represents a group of the formula (IXb), (IXc), (IXd) or (IXe).

[76] The compound described in [24] to [59], wherein U represents a group of the formula (VIIIa) to (VIIIe).

[77] The compound described in [24] to [46], wherein R³¹ represents an H atom, or a group —(CH₂)ₘ—NH—(CH₂)ₕ—CH₂—R¹², wherein R¹² represents a phosphonic acid ester group of the formula —P(═O)(OV)₂ or P(═O)(OV)(OH); each V independently is a methyl, ethyl, cyclohexyl or benzyl group; m is 1, 2, 3 or 4; and h is 0, 1, 2, or 3; provided that for the sum of m and h: 2≦x≦5.

[78] The compound described in [77], wherein R⁴⁷ in each case independently is an H atom; or a side chain of the amino acid lysine, ornithine, or arginine;

t=0, 1, 2, 3, 4, 5, 6, 7 or 8;

L represents OH, OEt, NH₂ or —NHNH₂;

U represents a group of the general formula (VII):

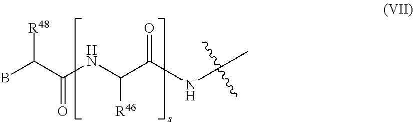

wherein B represents an H atom, a phenyl group, or a substituted phenyl group, substituted with 1 to 3 substituents, selected from the group comprising OH, F, Cl, Br, I and NO₂; R⁴⁸ is an H atom; and

(i) each R⁴⁶ in each case independently of each other represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; and s is 0, 1, 2, 3, 4, 5, 6, 7 or 8; or

(ii) each R⁴⁶ in each case independently of each other represents an H atom, or a group of the formula (IXb) to (IXe); and s=1, 2, 3 or 4.

[79] The compound described in [24], [25], and [27] to [78], provided that for the sum of all repeat units Y_{d}, Z_{f}, Y_{g}, and Zⱼ in the general formula (VI): 7≦x≦25.

[80] The compound described in [24], [25], and [27] to [78], provided that for the sum of all repeat units Y_{d}, Z_{f}, Y_{g}, and Zⱼ in the general formula (III) or (VI): 7≦x≦22.

[81] The compound described in [77] to [80], wherein each R¹¹ in each case represents a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

[82] The compound described in [77] to [81], wherein each R³¹ in each case represents an H atom, or a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

The compounds of the general formula (III) and (VI), as described above in [23] to [76] or [77] to [82], contain at least a group Z. Accordingly, the compounds according to the invention described in [23] to [76] or [77] to [82] of the general formula (III) and (IV) at the binding site of R¹¹ to the basic structure have at least 1 asymmetric centre (#). This asymmetric centre (#) at the binding site of R¹¹ to the basic structure preferably has the R-configuration.

If in the compounds of the general formula (III) and (VI), as described above in [23] to [76] or [77] to [82], two or more of these asymmetric centres (#) are present, at least 50% of the asymmetric centres (#), preferably 66%, 70%, 75% or 80%, more preferably 85%, 90% or 95%, and most preferably 100% have the R-configuration.

Alternatively, in the compounds of the general formula (III) and (VI) with two or more asymmetric centres (#), as for example described in [23] to [76] or [77] to [82], at least 50%, preferably 66%, 70%, 75% or 80%, more preferably 85%, 90% or 95%, and most preferably 100% of these asymmetric centres (#) have the S-configuration.

According to the invention a pharmaceutical composition is also disclosed, containing at least one (or more) oligomeric compound(s) according to the invention, and optionally at least one carrier, if necessary in combination with normal pharmacologically tolerated inactive ingredients and/or fillers, and/or at least one adjuvant.

The use of an oligomeric compound according to the invention as a medicine or drug is also an object of this invention. Generally, compounds according to the invention are administered using known and acceptable methods, either individually or in combination with any other therapeutic means. Administration can, for example be in one of the following ways: orally, e.g. as dragees, coated tablets, pills, semi-solids, soft or hard capsules, solutions, emulsions or suspensions; parenterally, e.g. as an injectable solution; rectally as suppositories; by inhalation, e.g. as a powder formulation or spray, transdermally or intranasally. For the preparation of such tablets, pills, semi-solids, coated tablets, dragees and hard gelatine capsules, the therapeutically usable product can be mixed with. pharmacologically inert, inorganic or organic carriers, for example lactose, sucrose, glucose, gelatine, malt, silica gel, starch or derivatives thereof, talc, stearic acid or its salts, dried skimmed milk and the like. For the production of soft capsules, carriers such as, for example, vegetable oils, liquid paraffin, animal or synthetic oils, wax, fat, and/or polyols can be used. For the preparation of liquid solutions and syrups, carriers such as, for example, water, alcohols, aqueous salt. solution, aqueous dextroses, polyols, glycerol, vegetable oils, liquid paraffin, animal and/or synthetic oils can be used. For suppositories, carriers such as, for example, vegetable oils, liquid paraffin animal and/or synthetic oils, wax, fat and/or polyols may be used. For aerosol formulations, compressed gases suitable for that purpose such as, for example, oxygen, nitrogen, chlorofluorocarbons, fluorinated hydrocarbons, chlorinated. hydrocarbons and carbon dioxide, may be used. The pharmaceutically usable agents may also contain additives for preservation, for stabilisation, emulsifiers, sweeteners, flavourings, salts for modifying the osmotic pressure, buffers, encapsulating additives and/or antioxidants.

Through its ability to bond to complementary nucleic acid sequences, an oligomeric compound or pharmaceutical composition according to the invention can be suitable for use in preventing and/or treating many different diseases. Examples of such diseases, which can be prevented with the oligomeric compounds according to the invention, or which can be treated with these are: diseases caused by viruses, such as for example human immunodeficiency virus (HIV), hepatitis B virus and hepatitis C virus or human papilloma virus (HPV); cancers, such, for example, skin cancer, lung cancer, liver cancer, prostate cancer, leukaemia, or brain tumours, rare neuromuscular diseases, such as, for example, Duchenne muscular dystrophy or spinal muscular atrophy; inflammatory diseases such as, for example, asthma, rheumatoid arthritis, or psoriasis; autoimmune diseases such as, for example, Crohn's disease or multiple sclerosis; neurological diseases such as, for example Parkinson's; or metabolic conditions such as, for example, high cholesterol or obesity.

The oligomeric compounds according to the invention, i.e. oligomeric compounds of the general formula (III) or (VI) (also referred to herein as N-phos oligomers), compared to the oligomeric compounds disclosed by EP 2041161 with alkyl-phosphonic acid ester groups (hereinafter referred to as: EP2041161 oligomers), demonstrate surprising and improved properties such as, for example, significantly improved bioavailability and a longer half-life in various therapeutically relevant organs. This was observed, for example, in a comparative tissue distribution study, in which mice were administered the respective oligomeric compounds and the quantities of these measured at different times in 18 therapeutically relevant organs (see Example 14 and FIGS. 1 and 2).

It has also been observed that the N-phos oligomers, compared to the EP 2041161 oligomers, have a stronger bonding to blood plasma proteins (see Example 15); this is advantageous to bioavailability and extends the half-life.

Compared to the EP 2041161 oligomers, the N-phos oligomers according to the invention also have a significantly improved, sequence independent water solubility (see Example 16).

It has furthermore been observed that the N-phos oligomers according to the invention have better bonding properties to DNA (higher melting point) (see Example 17).

The N-phos oligomers according to the invention also demonstrate a surprising significantly greater effect on the modulation of the gene expression compared to the EP 2041161 oligomers. The greater effect on the modulation of the gene expression is reflected, by way of example, in the down-regulation of NFkB in HeLa cells (see Example 18) and in the splice site modulation of the TNFR2 gene in THP1 cells (see Example 19).

An efficacy comparison between an N-phos oligomer according to the invention, an EP 2041161 oligomer and a U.S. Pat. No. 5,719,262 oligomer in the splice site modulation of the target TNFR2 in THP1 cells confirmed the significantly greater effect of the N-phos oligomers according to the invention on the modulation of the gene expression. The U.S. Pat. No. 5,719,262 oligomer had almost no effect, which is consistent with the observations of R. Corradini et al. (Current Topics in Medicinal Chemistry, 11 (12), pp. 1535-1554, (2011)), that the U.S. Pat. No. 5,719,262 oligomers demonstrate a strong tendency to accumulate within cells in vesicles and thus are not available in sufficient quantity for an antisense effect at the target site, of the mRNA in the cytosol or in the nucleus, (see Example 21 and FIG. 3).

The powerful effect of the N-phos oligomers on the modulation of the gene expression in the living organism is also apparent from the splice site modulation of the TNFR2 gene in the spleen and in the lymph nodes of the mouse (see Example 20). A more powerful effect could also be observed in the lungs (see Example 22 and FIG. 4 and Example 23 and FIG. 5), the kidneys (see Example 23 and FIG. 5 and Example 24 and FIG. 6), the liver (see Example 23 and FIG. 5) and in the muscles (see Example 25 and FIG. 7) for various N-phos oligomers according to the invention. In the kidneys of the mouse, N-phos oligomers according to the invention, by way of example, demonstrate an up to 12.6 times more powerful effect in the splice site modulation of the TNFR2 gene compared to the EP 2041161 oligomers (see Example 24 and FIG. 6). In the muscle of the mouse a more powerful effect of the N-phos oligomers according to the invention on the modulation of the gene expression of the dystrophin gene (exon 23 skipping) was demonstrated (see Example 25 and FIG. 7).

The targets NF-kappaB and TNFR2 play an important role in the TNF-α signal transduction pathway in immune cells. Spleen and lymph nodes are important organs of the immune system. So the oligomeric compounds (N-phos oligomers) according to the invention are suitable for therapeutic use in immune system mediated diseases such as for example inflammatory diseases, autoimmune diseases or cancer.

The monomers according to the invention of the general formula (I) can be prepared by means of reactions known to a person skilled in the art. By way of example, a monomer according to the invention of the general formula (I), wherein the asymmetric centre (#) has the R-configuration and R³ is a Cbz protective group, can be prepared according to the following synthesis schema (for a more detailed description see Examples 1-10):

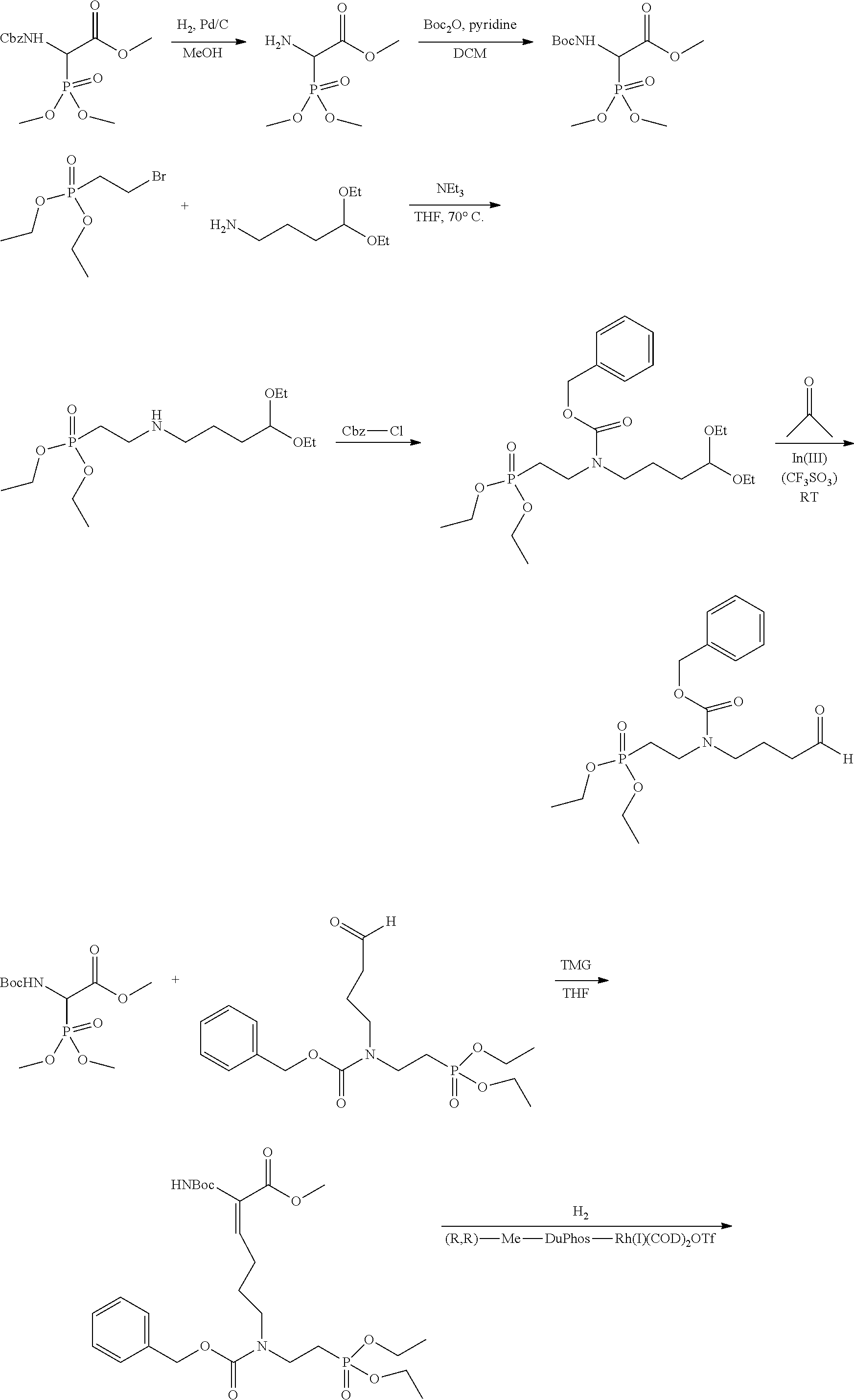

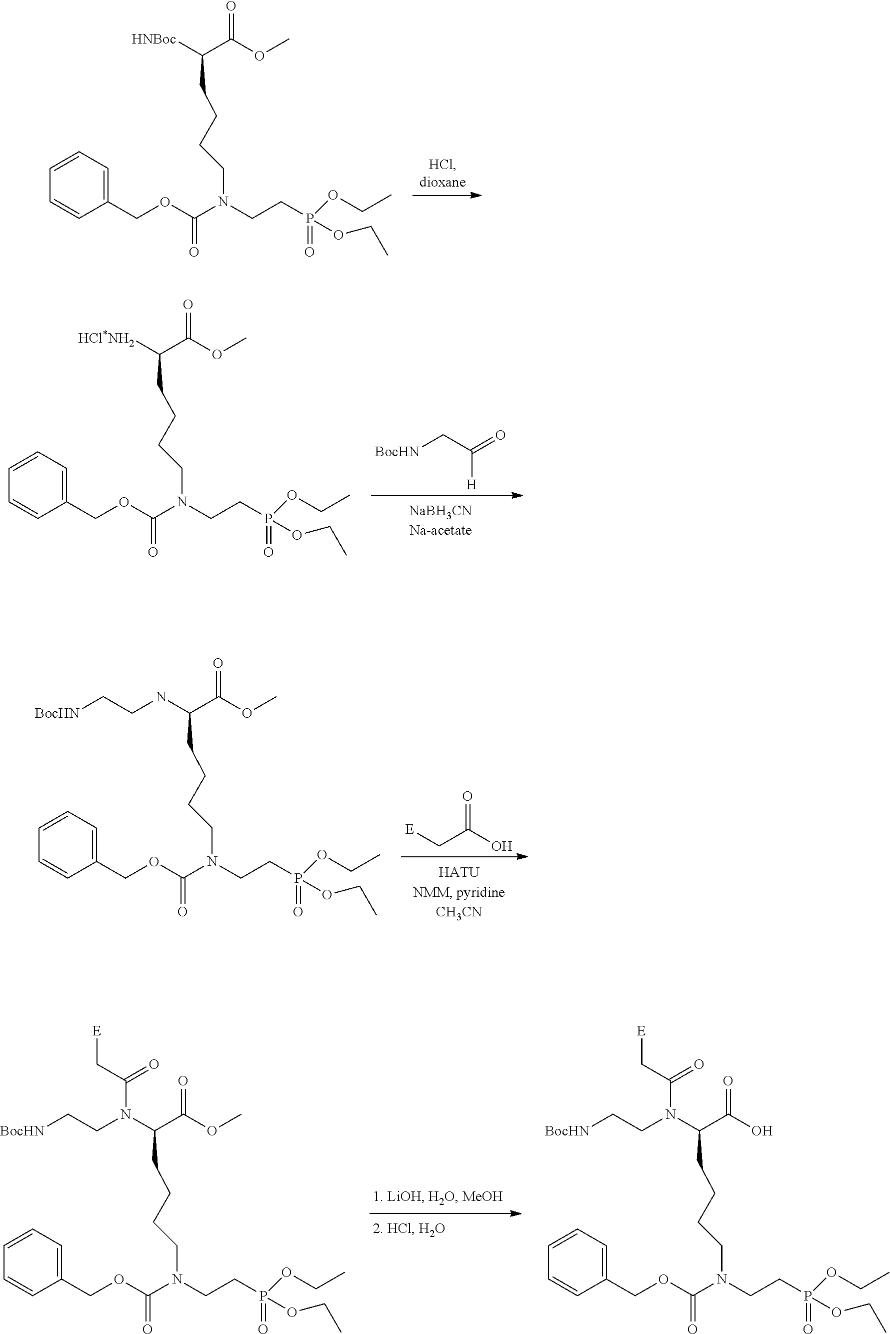

For the preparation of a monomer according to the invention of the general formula (I) (herein also referred to as N-Phos monomer) with an S-configuration at the asymmetric centre (#), during hydrogenation, instead of the (R,R)-Me-DuPhos-Rh(I)(OD)₂OTf catalyst, the (S,S)-Me-DuPhos-Rh(I) (COD)₂OTf catalyst is used.

The oligomeric compounds according to the invention with the general formula (III) or (VI), can be produced, by way of example, by means of the methods described in the literature by reacting monomers according to the invention of the general formula (I), or possibly further PNA monomers or amino acids in an in itself known manner (e.g. L. Christensen, R. Fitzpatrick, B. Gildea, K. H. Petersen, H. F. Hansen, T. Koch, M. Egholm, O. Buchardt, P. E. Nielsen, J. Coull, R. H. Berg, J.Pept.Sci. 3, 1995, 175-183. T. Koch, H. F. Hansen, P. Andersen, T. Larsen, H. G. Batz, K. Otteson, H. Örum, J. Pept.Res. 49, 1997, 80-88. F. Bergmann, W. Bannwarth, S. Tam, Tetrahedron Lett. 36, 1995, 6823-6826). Following the solid phase synthesis, the protective groups are split, so that oligomeric compounds according to the invention, e.g. compounds of the general formula (IV), are obtained.

### DESCRIPTION OF THE FIGURES

FIG. 1: bioavailability of a ³H labelled N-Phos oligomer according to the invention and of a ³H labelled EP2041161 oligomer in various tissues over a period of 14 days. As can be seen from the figure, the bioavailability of the ³H labelled N-Phos oligomer compared to the bioavailability of the ³H labelled EP2041161 oligomer over the period of 14 days is 1.7-4.6 times greater in all tissues.

FIG. 2: half-life of a ³H labelled N-Phos oligomer according to the invention and of a ³H labelled EP2041161 oligomer over a period of 14 days. FIG. 2 shows that the half-life of the ³H labelled N-Phos oligomer compared to the half-life of the ³H labelled EP2041161-oligomer over a period of 14 days is greater in most tissues, in the spleen actually by 2 times.

FIG. 3: Efficacy comparison between an N-Phos oligomer according to the invention, an EP 2041161 oligomer and a U.S. Pat. No. 5,719,262 oligomer in the splice site modulation of the target TNFR2 (exon 7 skipping) in THP1 cells. FIG. 3 shows that the N-Phos oligomer compared to the EP 2041161 oligomer has a 2.6 times stronger effect in the splice site modulation of the target TNFR2 in THP1 cells, while the modulation of the target TNFR2 in THP1 cells by the U.S. Pat. No. 5,719,262 oligomer is virtually zero.

FIG. 4: Effect of N-phos oligomers according to the invention with different radicals U, on the splice site modulation of the target TNFR2 (exon 7 skipping) in the lungs of mice. FIG. 4 shows that when N-phos oligomers according to the invention with formula (VI) having a radical U according to the general formula VII and a group of the formula IXc (cholesterol derivate) or IXd (folic acid derivative) are used as R⁴⁶ the effect on the gene expression in the splice site modulation is increased 560 times (cholesterol derivative) or 378 times (folic acid derivative) compared to the PBS negative control.

FIG. 5: Effect of the N-phos oligomers according to the invention N-Phos 23-1, N-Phos 23-2, N-Phos 23-3 and N-Phos 23-4 on the splice site modulation of the target TNFR2 (exon 7 skipping) in the kidneys, liver and lungs of mice. The N-phos oligomers according to the invention N-Phos 23-1, N-Phos 23-2, N-Phos 23-3 and N-Phos 23-4 differ in the nucleobase sequence, the radicals U, and the number and position of the groups of the general formula (IV) and (V) according to the general formula (VI). FIG. 5 shows that the N-phos oligomers according to the invention N-Phos 23-1, N-Phos 23-2, N-Phos 23-3 and N-Phos 23-4 in various mouse tissues (kidneys, liver and lungs) demonstrate very powerful effects on the gene expression of the mRNA isoform without exon 7. In the kidneys, by way of example, the effect of N-Phos 23-1 is increased 1,983 times compared with the PBS negative control.

FIG. 6: Efficacy comparison between N-phos oligomers according to the invention and EP 2041161 oligomers in the splice site modulation of the target TNFR2 (exon 7 skipping) in the kidneys of mice. The oligomers tested differ firstly by the sum of all repeat units Yd, Zf, Yg, and Zj (15 or 14) and secondly by the number and position of the groups of the general formula (IV) and (V). FIG. 6 shows that the effect of the N-phos oligomers on the gene expression of the mRNA isoform without exon 7 in a direct comparison with the EP2041161 oligomers is 12.6 or 6.7 times more powerful.

FIG. 7: In vivo effect of an N-phos oligomer according to the invention with 20 building blocks (sum of all repeat units Yd, Zf, Yg, and Zj according to the general formula (VI)=19) on the splice site modulation of the target dystrophin (exon 23 skipping) in the muscle of mdx mice. FIG. 7 shows that the N-Phos oligomer according to the invention in just a short-time experiment over 15 days with only 3 injections into the muscle, demonstrates a 9 times more powerful effect on the gene expression of the mRNA isoform without exon 23 compared with the PBS control group.

### EXAMPLES

Example 1

### Preparation of Compound 1

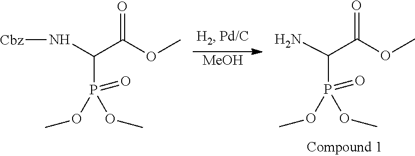

Description: 66.52 g (200 mmol) of 2-N-Cbz-amino-2-(dimethoxyphosphoryl)-acetic acid methyl ester in 300 ml of methanol are mixed with 2.13 g Pd/C 10% (corresponding to 1 Mol % Pd) and agitated under hydrogen pressure of 2 bar for 24 hours at room temperature. The catalyst is filtered off through Celite, and then the solvent is evaporated off from the filtrate. The product obtained is a light yellow oil, which when allowed to stand turns into a wax-like solid.

Yield: 39 g, 99%. ¹H-NMR (DMSO-d₆): 4.01 (d, 1H), 3.66-3.73 (m, 9H), 2.4-2.5 (s, br, 2H). ³¹P-NMR (DMSO-d₆): 23.6 ppm.

### Example 2

### Preparation of Compound 2

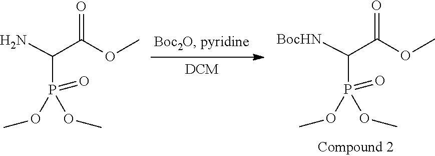

Description: 39 g (198 mmol, 1 eq) of compound 1 are dissolved in 1,000 ml of dichloromethane. 56.178 g (257 mmol, 1.3 eq) of Boc₂O and 16.1 ml (198 mmol, 1 eq) of pyridine are added. The mixture is agitated for 48 hours at room temperature. The solvent is removed in the rotary evaporator, and the residue is absorbed in acetic acid and washed with 5% citric acid solution, saturated sodium carbonate solution and saturated saline solution. Magnesium sulphate is then used for drying and evaporation. The remaining product is purified using flash chromatography (silica gel, hexane/acetic ether 1:5). The result is a yellow oil.

Yield: 43.5 g (146 mmol, 74%). ¹H-NMR (CDCl₃): 5.35 (d, br, 1H), 4.88 (dd, 1H), 3.80-3.86 (m, 9H), 1.46 (s, 9H). ³¹P-NMR (CDCl₃): 20.1 ppm

### Example 3

### Preparation of Compound 3

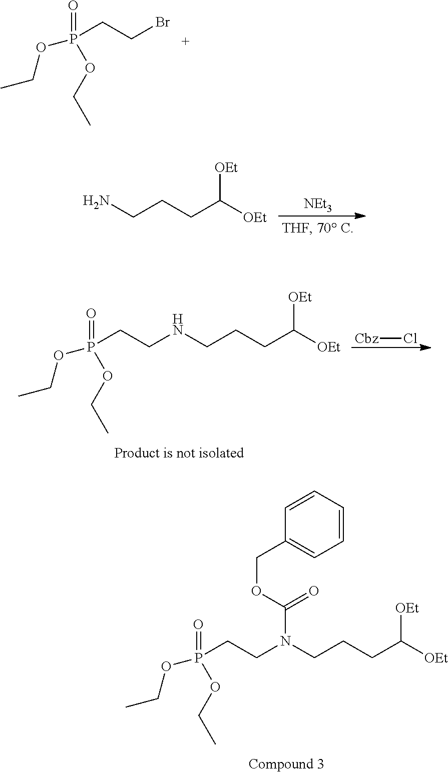

Description: 50 g (319 mmol, leq) of 4-aminobutyraldehyde diethyl acetal are dissolved at room temperature in 200 ml of THF and 51.5 ml (372 mmol, 1.2 eq) of triethylamine, and then 75.99 g (310 mmol, leq) of diethyl-2-bromethyl phosphonate are added in drops. Next the solution is heated to 70° C. and agitated at this temperature for 24 hours. The solvent is removed on the rotary evaporator. The residue is shaken vigorously with ether and filtered off. The remaining solid is extracted twice more with ether. The ether filtrates are combined and evaporated. The resulting product is a yellow oil, which is used without further purification in the next reaction.

¹H-NMR (DMSO-d₆): 4.45 (t, 1H); 3.98 (m, 4H); 3.54 and 3.42 (2m, 2×2H); 2.5-2.8 (m, 4H); 1.90 (m, 2H); 1.25-1.60 (m, 4H); 1.22 (t, 6H); 1.10 (2t, 6H). ³¹P-NMR (DMSO-d₆): 30 ppm.

The intermediate product obtained in the first step is dissolved in 400 ml of THF, mixed with 85.94 ml (620 mmol, 2 eq) of triethylamine and cooled to 0° C. Then 66.11 ml (465 mmol, 1.5 eq) of benzyl chloroformate are added in drops, the cooling is removed and agitation is performed overnight at room temperature. Next the reaction mixture is neutralised with 1 M of hydrochloric acid and the solvent is evaporated off. The residue is shaken with ether and stored overnight in the refrigerator. The resulting solid is separated and thoroughly washed twice more with ether. The ether solutions are combined and evaporated off. The residue is purified by chromatography via a silica gel column. In doing so, initially all impurities are eluted with hexane:acetic ether 2:1 and then the product with acetic ether.

Yield: 85.752 g (60.2%) colourless viscous oil. ¹H-NMR (DMSO-d₆): 7.34 (m, 5H); 5.07 (s, 2H); 4.44 (m, 1H); 3.94 (m, 4H); 3.6-3.2 (m, 8H); 2.02 (m, 2H); 1.46 (m, 4H); 1.19 (m, 6H); 1.09 (m, 6H). ³¹P-NMR (DMSO-d₆): 28.93 and 28.57 ppm.

### Example 4

### Preparation of Compound 4

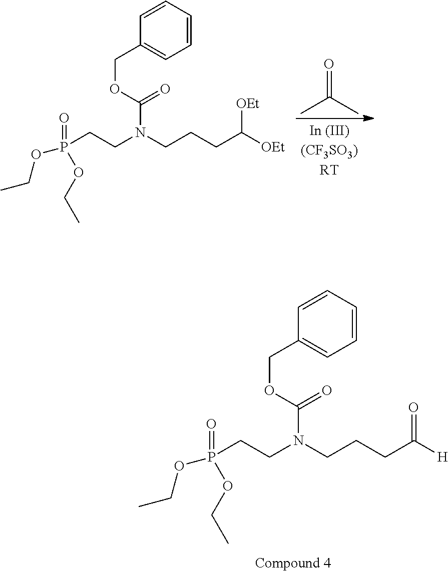

Description: 80 g of compound 3 in 1,000 ml of acetone are agitated with 0.98 g (1.74 mmol, 1Mol %) of indium (III) triflate at room temperature. The continuation of the equilibrium reaction is monitored using HPLC (RP₁₈, methanol/water 80:20). From time to time the solvent is evaporated off and replaced by fresh acetone. This takes place until the reaction is more than 95% complete. The solvent is then evaporated off. The substance, a yellow oil, is dried briefly in the high vacuum and immediately put to further use.

¹H-NMR (DMSO-d₆): 9.62 (d, 1H); 7.36 (m, 5H); 5.07 (s, 2H); 3.94 (m, 4H); 3.37 and 3.24 (2m, 4H); 2.41 (m, 2H), 2.04 (m, 2H); 1.73 (m, 2H); 1.22 (t, 6H). ³¹P-NMR (DMSO-d₆): 29.51, 29.14 ppm

### Example 5

### Preparation of Compound 5

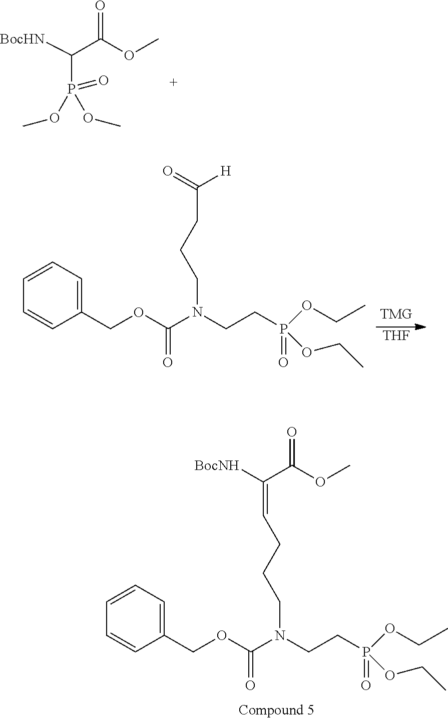

Description: In an argon atmosphere 29.438 g of compound 2 are cooled in 350 ml of THF to −70° C., then 12.96 ml (103 mmol, 1.04 eq) of N,N,N′N′-tetramethylguanidine are added in drops. After 10 minutes of agitation at −70° C., 38.170 g (99.04 mmol, 1 eq) of compound 4 in 60 ml of THF are added in drops. Agitation continues for a further hour at −70° C., and then the preparation is allowed to slowly reach room temperature and is agitated overnight.

The solvent is evaporated off. The residue is dissolved in approximately 400 ml of acetic ether, washed twice with 5% citric acid solution and once with saturated NaCl solution, dried with magnesium sulphate and evaporated off. A yellow oil is obtained.

Yield: 53,228 g, 95.6 mmol, 96.5%. ¹H-NMR (CDCl₃): 7.33 (s, 5H); 6.48 (t, 1H); 5.13 (s, 2H); 4.08 (m, 4H); 3.81 and 3.76 (2s, total 3H); 3.49 (m, 2H); 3.30 (t, 2H); 2.21 (m, 2H); 2.04 (m, 2H); 1.71 (m, 2H); 1.48 and 1.46 (2s, total 9H); 1.28 (m, 6H). ³¹P-NMR (CDCl₃): 29.72 and 29.15 ppm.

### Example 6

### Preparation of Compound 6

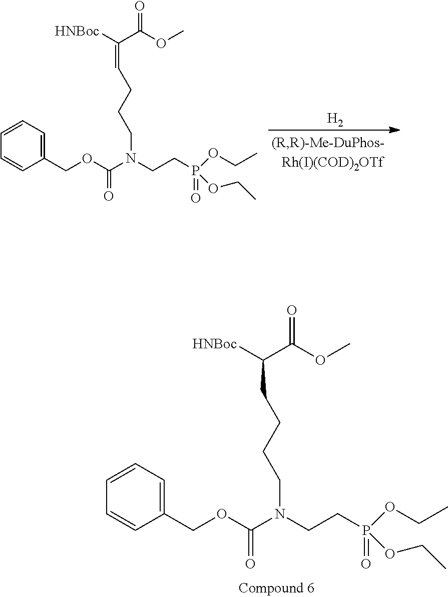

Description: In the reaction bottle of a Parr hydrogenation apparatus, under argon 450 mg (0.96 mmol, 1Mol %) of Bis(1,5-cyclooctadienyl)rhodium(I)-trifluormethane sulphonate and 306 mg (0.96 mmol, 1Mol %) of (−)-1,2-Bis-[(2R, 5R)-2,5-dimethyl-phospholano]-benzene are dissolved in approximately 50 ml of methanol, then 53.228 g (96 mmol) of compound 5, dissolved in 250 ml of methanol, are added. The bottle is mounted on the hydrogenation apparatus, evacuated three times and filled with hydrogen. Finally, a hydrogen pressure of 4.5-5 bar is set and the bottle agitated for 24 hours.

The excess hydrogen is let off, the bottle is removed and the reaction solution is filtered through Celite. The filtrate is evaporated off and dried in the vacuum. A light brown oil is obtained.

Yield: 53.712 g, 96 mmol, quantitative. ¹H-NMR (CDCl₃): 7.35 (m, 5H); 6.48 and 6.35 (2m, total 1H); 5.13 (s, 2H); 4.27 (m, 1H); 4.07 (m, 4H); 3.73 (s, 3H); 3.48 (m, 2H); 3.27 (m, 2H); 2.25-1.95 (m, 4H); 1.75-1.55 (m, 4H); 1.45 (s, 9H); 1.28 (m, 6H). ³¹P-NMR (CDCl₃): 29.78 and 29.23 ppm.

### Example 7

### Preparation of Compound 7

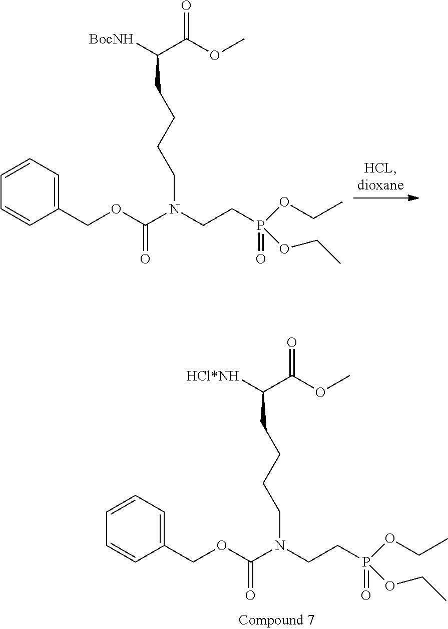

Description: 240 ml of a 4M solution of HCl in dioxane are added in drops to 53.94 g (96.6 mmol) of compound 6 in 120 ml of THF. Agitation is then performed at room temperature. The reaction process is monitored by means of HPLC (methanol/water 70:30). After 2-3 hours the Boc splitting is complete. The solvent is evaporated off and the residue washed with diethyl ether and dried. A stodgy brown oil is obtained.

Yield: 47.83 g, quantitative. ¹H-NMR (DMSO-d₆): 8.64 (s, br, 2H); 7.36 (m, 5H); 5.07 (s, 2H); 3.97 (m, 5H); 3.73 (s, 3H); 3.38 (m, 2H); 3.22 (m, 2H); 2.04 (m, 2H); 1.80 (m, 2H); 1.48-1.35 (m, 4H); 1.22 (m, 6H). ³¹P-NMR (DMSO-d₆): 29.012 and 28.62 ppm.

### Example 8

### Preparation of Compound 8

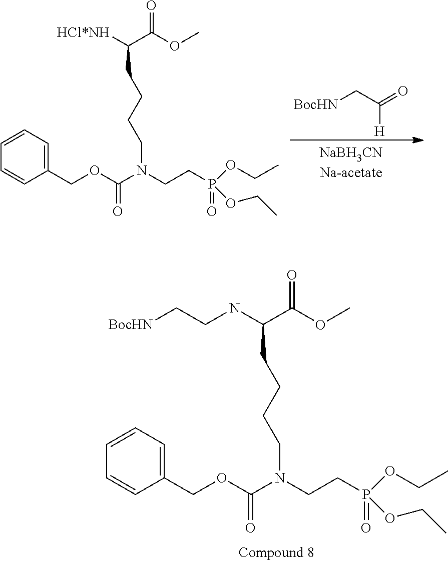

Description: 16.65 g (29.8 mmol, leq.) of compound 7 in 100 ml of methanol are cooled to 0° C. and mixed with 5.414 g (66 mmol, 2.2 eq) of sodium acetate. Then 5.218 g (32.8 mmol, 1.1 eq) of N-Boc aminoacetaldehyde in 150 ml methanol are added in drops. Agitation is performed for one hour at 0° C., then 2.074 g (33 mmol, 1.1 eq) of sodium cyanoborohydride are added in portions. Once the gas development has abated, the cooling bath is removed and agitation is performed overnight at room temperature.

The solvent is removed in the rotary evaporator, the residue absorbed in acetic ether and washing performed with sodium hydrogen carbonate solution (semi-saturated) and saturated sodium chloride solution. The organic phase is dried with magnesium sulphate, evaporated and vacuum-dried. The raw product is purified via a silica gel column (dichloromethane/methanol (5%, v/v)). A viscous, yellow oil is obtained.

Yield: 15.379 g (25.6 mmol, 86%). ¹H-NMR (DMSO-d₆): 7.36 (m, 5H); 6.69 (m, 1H); 5.06 (s, 2H); 4.02 (m, 4H); 3.4-3.15 (3m, total 7H); 2.95 (m, 1H); 2.38 (m, 1H); 2.01 (m, 2H); 1.60-1.30 (m, 6H); 1.38 (s, 9H); 1.20 (m, 6H). ³¹P-NMR (DMSO-d₆): 29.00 and 28.60 ppm.

### Example 9

### Preparation of Compound 9

E=Cbz-A, Cbz-C, Cbz-G, T

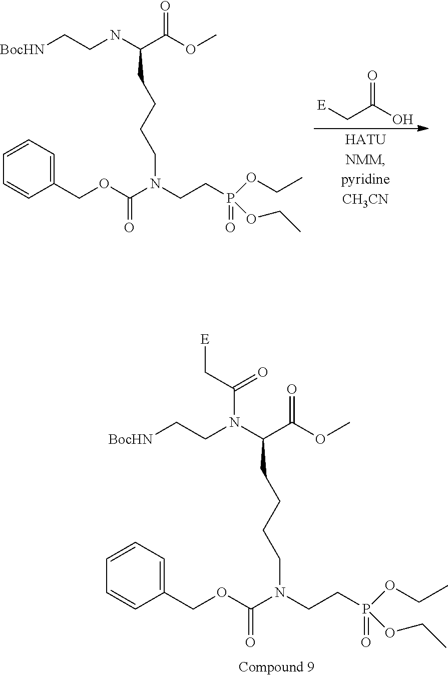

Description: 20.25 mmol (1.5 eq) of acetic acid components (N6-Cbz-adenin-9-yl-acetic acid, N4-Cbz-cytosin-l-yl-acetic acid, N2-Cbz-guanin-9-yl-acetic acid or thymidin-l-yl-acetic acid) in 70 ml of acetonitrile, 20 ml of pyridine and 4.5 ml (40, 5 mmol, 3 eq) of N-methylmorpholine are cooled to 0° C. and mixed with 7.186 g (18.9 mmol, 1.4 eq) of HATU. The cooling is removed, and agitation is performed for 10 minutes at room temperature. The mixture is then added slowly to 8.1 g (13.5 mmol, leq) of compound 8, dissolved in 50 ml of acetonitrile. Agitation is performed for 1 hour at room temperature, and then overnight at 40° C.

The mixture is cooled again to room temperature and diluted with 20 ml of water. Following agitation for 30 minutes the solvent is evaporated off. The residue is evaporated off twice more with dichloromethane, in order to remove as much pyridine as possible. The residue is then absorbed again in dichloromethane and placed in the refrigerator overnight. The resulting solid is filtered off, and the filtrate evaporated off and purified by means of flash chromatography (silica gel, 2-5% methanol in dichloromethane), wherein the product is obtained as a white-yellow foam.

Compound 9, E=Cbz-A:

Yield: 62%. ¹H-NMR (DMSO-d₆): 10.65 (s, 1H); 8.59 (s, 1H); 8.35 and 8.29 (2s, total 1H); 1.47-7.24 (m, 10H); 6.90 and 6.72 (2m, total 1H); 5.42-5.00 (m, 6H); 3.92 (m, 5H); 3.56 and 3.49 (2s, total 3H); 3.50-2.95 (m, 8H); 2.12-1.30 (m, 8H); 1.38 and 1.35 (2s, total 9H); 1.16 (m, 6H); ³¹P-NMR (DMSO-d₆): 29.52 and 29.16 ppm.

Compound 9, E=Cbz-C:

Yield: 59%. ¹-NMR (DMSO-d₆): 10.77 (s, br, 1H); 7.94 (d, ¹H), 7.42-7.33 (m, 10H); 7.01 (d, 1H); 6.91 and 6.80 (2m, total 1H); 5.19 (S, 2H); 5.07 (s, 2H); 4.80-4.6 (m, 2H); 4.36 (m, 1H); 3.94 (m, 4H); 3.7 and 3.59 (2s, total 3H); 3.5-2.8 (m, 8H); 2.05-1.3 (m, 8H); 1.38 and 1.37 (2s, total 9H); 1.19 (m, 6H). ³¹P-NMR (DMSO-d₆): 29.02 and 28.63 ppm.

Compound 9, E=Cbz-G:

Yield: 77.5%. ¹-NMR (DMSO-d₆): 11.33 (s, br, 2H); 7.85 (s, 1H); 7.45-7.30 (m, 10 H); 6.99 and 6.81 (2m, total 1H); 5.26 (s, 2H); 5.06 (m, 4H); 4.58 and 4.31 (2m, total 1H); 3.934 (m, 4H); 3.56 (s, 3H); 3.31-3.19 (m, 8H); 2.21-1.30 (m, 8H); 1.36 and 1.35 (2s, total 9H); 1.19 (m, 6H). ³¹P-NMR (DMSO-d₆): 28.98 and 28.59 ppm.

Compound 9, E=T:

Yield: 78% ¹-NMR (DMSO-d₆): 11.26 (s, br, 1H); 7.35 (m, 6H); 6.90 and 6.79 (2m, total 1H); 5.07 (s, 2H); 4.63-4.49 (m, 2H); 4.31 (m, 1H); 3.94 (m, 4H); 3.70 and 3.58 (2s, total 3H); 3.46-3.12 (m, 8H); 2.11-1.30 (m, 8H); 1.76 (s, 3H); 1.38 and 1.35 (2s, total 9H); 1.19 (m, 6H). ³¹P-NMR (DMSO-d₆): 29.61 and 29.25 ppm.

### Example 10

### Preparation of E=Cbz-A, Cbz-C, Cbz-G, T

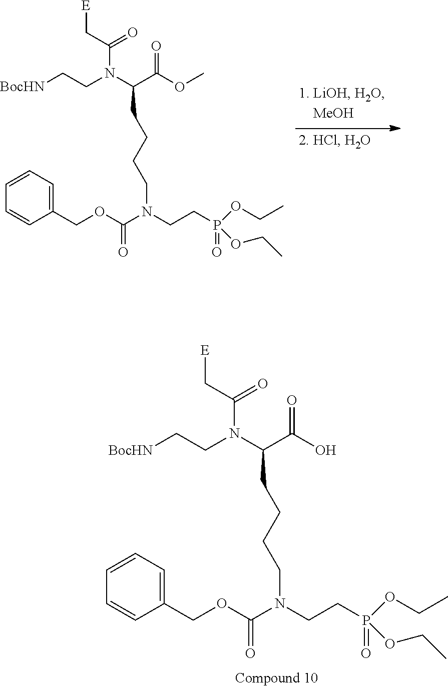

Description: 2.31 mmol of compound 9 (E=Cbz-A, Cbz-C, Cbz-G or T) are cooled in 12 ml of water/methanol 1:1 to 0° C., then 12 ml of a 2N NaOH solution are added in drops. Agitation is performed for 15 minutes at 0° C., and then at room temperature until the saponification is complete according to DC control (silica gel, 10% methanol in dichloromethane) (duration: approximately 1 hour). A little water is then used for dilution and any undissolved substances present are briefly centrifuged off. The clear solution is diluted to approximately 300 ml with water and cooled again to 0° C. The pH is adjusted to 2.5 with 1M of HCl solution, resulting in the precipitation of a white solid. The solution is extracted with dichloromethane (approximately 5 times), until no further product passes to the organic phase (DC control). The combined organic phases are dried with magnesium sulphate, evaporated off and vacuum dried. The product is obtained as a white-yellow solid.

Compound 10, E=Cbz-A:

Yield: 72%. ¹-NMR (DMSO-d₆): 10.68 (s, br, 1H); 8.59 (s, br, 1H); 8.35 and 8.29 (2s, br, total 1H); 7.49-7.33 (m, 10 H); 6.98 and 6.85 (2m, total 1H); 5.35-5.04 (m 6H); 4.62 and 4.22 (2m, total 1H,); 3.93 (m, 4H,); 3.50-2.85 (m, 8H); 2.20-1.30 (m, 8H); 1.39 and 1.36 (2s, total 9H); 1.17 (m, 6H). ³¹P-NMR (DMSO-d₆): 29.55 and 29.19 ppm.

Compound 10, E=Cbz-C:

Yield: 59%. ¹H-NMR (DMSO-d₆): 7.95 (d, 1H); 7.42-7.28 (m, 10H); 7.01 (d, 1H); 6.90 and 6.85 (2m, total 1H); 5.19 (s, 2H); 5.07 (s, 2H); 4.81-4.65 (m, 2H); 4.33 (m, 1H); 3.933 (m, 4H); 3.45-3.15 (m, 8H); 2.15-1.30 (m, 8H); 1.38 and 1.35 (2s, total 9H); 1.20 (m, 6H). ³¹P-NMR (DMSO-d₆): 29.03 and 28.66 ppm.

Compound 10, E=Cbz-G:

Yield: 62%. ¹-NMR (DMSO-d₆): 11.43 (s, br, 1H); 11.33 (s, br, 1H); 7.85 (s, 1H); 7.44-7.31 (m, 10H); 6.97 and 6.81 (2m, total 1H); 5.24 (s, 2H); 5.10-5.00 (m, 4H); 4.51 and 4.28 (2m, total 1H); 3.91 (m, 4H); 3.52-3.08 (m, 8H);2.12-1.28 (m, 8H); 1.36 and 1.34 (2s, total 9H); 1.15 (m, 6H). ³¹P-NMR (DMSO-d₆): 29.61 and 29.21 ppm.

Compound 10, E=Cbz-T:

Yield: 72%. ¹-NMR (DMSO-d₆): 11.27 (s, br, 1H); 7.35 (m, 6H); 6.88 (m, 1H); 5.07 (s, 2H); 4.65-4.48 (m, 2H); 4.37 and 4.38 (2m, total 1H); 3.94 (m, 4H); 3.45-3.17 (m, 8H); 2.11-1.30 (m, 8h); 1.75 (s, 3H); 1.38 and 1.36 (2s, total 9H); 1.19 (m, 6H). ³¹P-NMR (DMSO-d₆): 29.64 and 29.26 ppm.

### Example 11

### Preparation of Oligomeric Compounds According to the Invention of the General Formulae (III) or (VI)

By sequential linking of appropriate compounds of the general formula (I) with monomer units selected from the group comprising N-acetyl-N-(2-aminoethyl)glycine building blocks, amino acids, amino acid derivatives, and a group B-CH(R⁴⁸)COOH, by means of solid phase peptide synthesis oligomers according to the invention are prepared.

To facilitate the representation of the units Z according to the general formula (V) in oligomeric compounds of the general formula (IV), by way of example the following abbreviations are used: T^{R}, C^{R}, G^{R}, A^{R}, P^{R}, T^{S}, C^{S}, G^{S}, A^{S} and P^{S}. Here T, C, G, A, and P (phenyl) in each case stands for the nucleobase of the respective monomer unit and the superscript R or S stands for the R-configuration or S-configuration at the asymmetric centre (#) of the unit Z according to the general formula (V).

Monomers, consisting of N-acetyl-N-(2-aminoethyl)glycine building blocks, are abbreviated analogously for the abovementioned monomers of the general formula (V), with the difference that instead of the capital letters for the nucleobase and the superscript letters for the configuration (e.g. A^{R}) the corresponding lower case letter a is used. By way of example, a monomer with C as nucleobase is abbreviated to c.

### Example 12

### Oligomeric Compounds of the General Formulae (III) or (VI) with 4-Fluorophenyl-Acetate Substituent as Group B-CH(R⁴⁸)COOH

For the preparation of the compounds according to the invention by means of solid phase peptide synthesis, the following synthesis protocol is used:

Step 1: 3 h pre-soak 10 mg of resin (MBHA resin, Novabiochem, Low Loaded approximately 0.5-06 mmol/g) in dichloromethane.

Step 2: Start synthesis cycle: 4× washing with dichloromethane.

Step 3: Neutralise the resin: 3× washing with dichloromethane/DIPEA (5%).

Step 4: 5× washing with dichloromethane.

Step 5: 5× washing with NMP.

Step 6: 1 min pre-activation of 4 equivalents of the correspondingly protected compound (compound of general formula (I)/PNA-monomer/amino acid/amino acid derivative) with 3.8 equivalents of HATU and 9 equivalents of NMM in NMP/pyridine (2:1).

Step 7: React the activated protected compound (compound of general formula (I)/PNA-monomer/amino acid/amino acid derivative) with the solid phase (1^{st} coupling; duration: 60 min).

Step 8: 4× washing with NMP.

Step 9: Repeat steps 6 to 8 (2^{nd} coupling).

Step 10: Check coupling efficiency with ninhydrin (Kaiser test; if the Kaiser test is positive, steps 6 to 8 must be repeated with the corresponding protected compound (compound of general formula (I)/PNA-monomer/amino acid/amino acid derivative)).

Step 11: After a negative Kaiser test, 1× capping with a solution of Ac₂O/NMP/pyridine (1:25:25) for 10 mins.

Step 12: 5× washing with NMP.

Step 13: Switch the solvent to dichloromethane: 5× washing with DCM.

Step 14: Boc splitting by reacting with TFA/m-cresol (95:5). Reaction time: 2× 3 min each

Step 15: 5× washing with DCM.

Step 16: Switch the solvent to NMP. 5× washing with NMP.

Step 17: Repeat the synthesis cycle (steps 6 to 16)—for coupling with the last correspondingly protected compound (compound of general formula (I)/PNA-monomer/amino acid/amino acid derivative). Next, repeat as necessary the synthesis cycle (steps 6 to 16)—for coupling with the last correspondingly protected compound (compound of general formula (I)/PNA-Monomer/amino acid/amino acid derivative).

Step 18: 5× washing with dichloromethane.

Step 19: Boc splitting by reacting with TFA/m-Kresol (95:5). Reaction time: 2× 3 min each.

Step 20: 5× washing with dichloromethane.

Step 21: 5× washing with NMP.

Step 22: 1 min pre-activate 6 equivalents of 4-fluorophenyl-acetic acid with 5.7 equivalents of HATU and 13 equivalents of NMM in NMP/pyridine (2:1).

Step 23: React activated 4-fluorphenyl-acetic acid with the solid phase (duration: 60 min).

Step 24: 4× washing with NMP.

Step 25: Repeating, as necessary, steps 23 to 24 (2nd coupling).

Step 26: 5× washing with dichloromethane.

Step 27: For drying: 5× washing with diethyl ether.

A compound of general formula (III) or (VI) is obtained, bonded to the C-terminal end of the resin.

Splitting of the compound according to the invention of the general formulae (III) or (VI) from the resin: The resin with the compound according to the invention is shaken in a solution of trifluoroacetic acid, trifluormethane sulphonic acid, thioanisole and ethane dithiol (85/12.5/1.7/0.8, v/v/v/v) for 2 hours. The liquid phase is filtered off and the raw product precipitated by addition of cold ether. The raw product is desalinated by size exclusion chromatography. The raw product is purified by preparative HPLC via an RP-C₁₈ column with methanol/water. The compound according to the invention is obtained as a colourless solid with a yield of approximately 50%. The mass of the compound according to the invention is characterised with HPLC-ESI.

### Example 13

### Examples of Prepared Compounds of the General Formulae (III) or (VI)

By following the general procedure of Example 12, oligomeric compounds of the general formulae (III) or (VI) are obtained, which are abbreviated as follows:

By way of example, an inventive oligomeric compound of monomers of the general formula (I) with an asymmetric centre with R-configuration and other PNA monomers and the amino acid L-lysine (abbreviated to: Lys^{L}) is prepared and in the final step the α-amino function of the lysine is capped with acetyl and finally the oligomeric compound is then split as a primary amide from the resin, abbreviated to Ac-Lys^{L}-cC^{R}gG^{R}tcgcaG^{R}cT^{R}gG^{R}-NH₂.

By way of example, an inventive oligomeric compound of monomers of the general formula (I) with an asymmetric centre with R-configuration and other PNA-monomers and the amino acid glycine (abbreviated to: Gly) is prepared and in the final step the α-amino function of the lysine is capped with phenyl acetate (abbreviated to: Pac) and finally the oligomeric compound is then split as a primary amide from the resin, abbreviated to Pac-Gly-agcccT^{S}aacT^{S}gcacT^{S}T^{S}ccaT^{S}-NH₂.

By way of example, an inventive oligomeric compound of monomers of the general formula (I) with an asymmetric centre with R-configuration and other PNA-monomers and the amino acid D-lysine (abbreviated to: Lys^{D}) is prepared and in the final step the α-amino function of the lysine is capped with 4-fluoro-phenyl acetate (abbreviated to: FluPac) and finally the oligomeric compound is then split as a primary amide from the resin, and then the ε-amino-function of the lysine is coupled to the fluorescent dye ATTO647, abbreviated to FluPac-Lys^{D}(ATTO647)-cC^{R}gG^{R}gG^{R}tcgcaG^{R}cT^{R}gG^{R}-NH₂.

Examples of other fluorescent dyes are ATTO, MegaRed, Alexa, BODIPY and TAMRA.

The following are examples of compounds according to the invention of the general formula (IV) that have been prepared:

Pac-Lys^{L}-agcccT^{R}aacT^{R}gcacT^{R}T^{R}ccaT^{R}-NH₂

Pac-Lys^{L}-T^{R}T^{R}ccaT^{R}ccT^{R}T^{R}ggagcT^{R}T^{R}ggcT^{R}-NH₂

Pac-Lys^{L}-cT^{R}aacT^{R}gcacT^{R}T^{R}ccaT^{R}ccT^{R}T^{R}-NH₂

Pac-Lys^{L}-T^{R}T^{R}cccagcccT^{R}aacT^{R}gcacT^{R}-NH₂

Pac-Lys^{L}-gacccT^{R}T^{R}cccagcccT^{R}aacT^{R}-NH₂

Pac-Lys^{L}-ggT^{R}agacccT^{R}T^{R}cccagcccT^{R}-NH₂

Pac-Lys^{L}-T^{R}T^{R}cgT^{R}ccaT^{R}ggccggggT^{R}cc-NH₂

Pac-Lys^{L}-T^{R}T^{R}cgT^{R}ccagT^{R}gccggggT^{R}cc-NH₂

FluPac-Lys^{L}-cC^{R}gG^{R}gG^{R}tcgcaG^{R}cT^{R}gG^{R}-NH₂

FluPac-Lys^{L}-cC^{R}gG^{R}gG^{R}tcccgG^{R}gG^{R}gC^{R}-NH₂

FluPac-Lys^{L}-CC^{R}aT^{R}gG^{R}ccgggG^{R}tC^{R}cC^{R}-NH₂

FluPac-Lys^{L}-GT^{R}tC^{R}gT^{R}ccatgG^{R}cC^{R}gG^{R}-NH₂

FluPac-Lys^{L}-GG^{R}gG^{R}gA^{R}acagtT^{R}cG^{R}tC^{R}-NH₂

FluPac-Lys^{L}-GA^{R}GG^{R}gG^{R}ggaacaG^{R}tT^{R}cG^{R}-NH₂

FluPac-Lys^{L}-CG^{R}gG^{R}aA^{R}gatgaG^{R}gG^{R}gG^{R}-NH₂

FluPac-Lys^{L}-AG^{R}aG^{R}gC^{R}ctgggC^{R}tG^{R}gC^{R}-NH₂

FluPac-Lys^{L}-cC^{R}aC^{R}aT^{R}aggggC^{R}cA^{R}gA^{R}-NH₂

FluPac-Lys^{L}-tT^{R}gG^{R}gC^{R}tgctcA^{R}aT^{R}gA^{R}-NH₂

FluPac-Lys^{L}-cG^{R}cG^{R}gA^{R}gcgccC^{R}cT^{R}cG^{R}-NH₂

FluPac-Lys^{L}-tG^{R}gG^{R}gT^{R}gggtcT^{R}tG^{R}gT^{R}-NH₂

FluPac-Lys^{L}-gT^{R}cG^{R}cT^{R}gtctcC^{R}gC^{R}tT^{R}-NH₂

FluPac-Lys^{L}-aG^{R}cT^{R}gA^{R}ccctgA^{R}aG^{R}tT^{R}-NH₂

FluPac-Lys^{L}-aG^{R}cT^{R}gA^{R}cctgcA^{R}aG^{R}tT^{R}-NH₂

FluPac-Lys^{L}-cC^{R}gG^{R}gG^{R}tcgcaG^{R}cT^{R}GA^{R}-NH₂

FluPac-Lys^{L}-gC^{R}cG^{R}gG^{R}gtcgcA^{R}gC^{R}tG^{R}-NH₂

FluPac-Lys^{L}-cC^{R}aT^{R}gG^{R}tcaggG^{R}tC^{R}cC^{R}-NH₂

FluPac-Lys^{L}-gC^{R}cT^{R}gG^{R}gctggC^{R}tC^{R}tG^{R}-NH₂

FluPac-Lys^{L}-cC^{R}aC^{R}aT^{R}aaggcC^{R}cA^{R}gA^{R}-NH₂

FluPac-Lys^{L}-tG^{R}gT^{R}gG^{R}tatctG^{R}tG^{R}cT^{R}-NH₂

FluPac-Lys^{L}-tT^{R}gA^{R}tC^{R}ttgatG^{R}gT^{R}gG^{R}-NH₂

FluPac-Lys^{L}-tG^{R}gG^{R}gT^{R}gggtcT^{R}tG^{R}gT^{R}-NH₂

FluPac-Lys^{L}-cC^{R}tatC^{R}aC^{R}gA^{R}ttagcA^{R}tT^{R}aA^{R}-NH₂

FluPac-Lys^{L}-cC^{R}catG^{R}gA^{R}aT^{R}tcagtT^{R}cT^{R}cA^{R}-NH₂

FluPac-Lys^{L}-cC^{R}tatC^{R}AC^{R}gA^{R}tatgcT^{R}aT^{R}aA^{R}-NH₂

Ac-Lys^{L}-cC^{R}gG^{R}gG^{R}tcgcaG^{R}cT^{R}gG^{R}-NH₂

TML-Lys^{L}-cC^{R}gG^{R}gG^{R}tcgcaG^{R}cT^{R}gG^{R}-NH₂ (TML=ε-trimethyl-lysine)

FluPac-Lys^{L}-gT^{R}cG^{R}cT^{R}gtctcC^{R}gC^{R}tT^{R}-NH₂

Ac-Lys^{L}-gT^{R}cG^{R}cT^{R}gtctcC^{R}gC^{R}tT^{R}-NH₂

FluPac-Lys^{L}-cC^{R}gG^{R}gG^{R}tgccaG^{R}cT^{R}gG^{R}-NH₂

FluPac-Lys^{L}-cC^{R}ggG^{R}gtgccA^{R}gctG^{R}g-NH₂

FluPac-Lys^{L}-cC^{R}aC^{R}aA^{R}tcagtC^{R}cT^{R}aG^{R}-NH₂

FluPac-Lys^{L}-cA^{R}gT^{R}cC^{R}tagaaA^{R}gA^{R}aA^{R}-NH₂

FluPac-Lys^{L}-aC^{R}tT^{R}tT^{R}cacctG^{R}gG^{R}tC^{R}-NH₂

FluPac-Lys^{L}-cA^{R}aT^{R}aC^{R}tattgC^{R}aC^{R}tG^{R}-NH₂

FluPac-Lys^{L}-gC^{R}tT^{R}tG^{R}acaatA^{R}cT^{R}aT^{R}-NH₂

FluPac-Lys^{L}-tG^{R}aC^{R}aA^{R}tactaT^{R}tG^{R}cA^{R}-NH₂

FluPac-Lys^{L}-aG^{R}tA^{R}tT^{R}ggaccC^{R}tT^{R}aC^{R}-NH₂

FluPac-Lys^{L}-gA^{R}aC^{R}aG^{R}tattgG^{R}aC^{R}cC^{R}-NH₂

FluPac-Lys^{L}-gA^{R}acaG^{R}tA^{R}tT^{R}ggaccC^{R}tT^{R}aC^{R}-NH₂

FluPac-Lys^{L}-tC^{R}aG^{R}tC^{R}tgataA^{R}gC^{R}tA^{R}-NH₂

FluPac-Lys^{L}-tC^{R}aA^{R}cA^{R}tcagtC^{R}tG^{R}aT^{R}-NH₂

FluPac-Lys^{L}-aC^{R}aT^{R}cA^{R}gtctgA^{R}tA^{R}aG^{R}-NH₂

FluPac-Lys^{L}-cC^{R}gG^{R}gG^{R}tC^{R}gC^{R}aG^{R}cT^{R}gG^{R}-NH₂

FluPac-Lys^{L}(ATTO647)-cC^{R}gG^{R}gG^{R}tcgcaG^{R}cT^{R}gG^{R}-NH₂

FluPac-Lys^{L}(MegaRed)-cC^{R}gG^{R}gG^{R}tcgcaG^{R}cT^{R}gG^{R}-NH₂

FluPac-Lys^{L}(ATTO647)-cC^{R}gG^{R}gG^{R}tC^{R}gC^{R}aG^{R}cT^{R}gG^{R}-NH₂

FluPac-Lys^{L}(MegaRed)-cC^{R}gG^{R}gG^{R}tC^{R}gC^{R}aG^{R}cT^{R}gG^{R}-NH₂

FluPac-gG^{R}ccaA^{R}aC^{R}cT^{R}cggctT^{R}aC^{R}cT^{R}-NH₂

FluPac-gG^{R}ccaA^{R}aC^{R}cT^{R}gcgctT^{R}aC^{R}cT^{R}-NH₂

FluPac-Lys^{L}(ATTO)-cC^{R}gG^{R}gG^{R}tcgcaG^{R}cT^{R}gG^{R}-NH₂

FluPac-Lys^{L}(ATTO)-cC^{R}gG^{R}gG^{R}tC^{R}gC^{R}aG^{R}cT^{R}gG^{R}-NH₂

FluPac-Lys^{L}(ATTO)-cC^{R}gG^{R}gG^{R}tgccaG^{R}cT^{R}gG^{R}-NH₂

Ac-Lys^{L}(ATTO)-cC^{R}gG^{R}gG^{R}tcgcaG^{R}cT^{R}gG^{R}-NH₂

Ac-Lys^{L}(ATTO)-cC^{R}gG^{R}gG^{R}tcgcaG^{R}cT^{R}g-NH₂

FluPac-Lys^{L}(ATTO)-gT^{R}cG^{R}cT^{R}gtctcC^{R}gC^{R}tT^{R}-NH₂

FluPac-Lys^{L}(ATTO)-cC^{R}ggG^{R}gtgccA^{R}gctG^{R}g-NH₂

FluPac-Lys^{L}(ATTO)-C^{R}cgggG^{R}tC^{R}gC^{R}agctgG^{R}-NH₂

FluPac-Lys^{L}(Alexa)-cC^{R}gG^{R}gG^{R}tcgcaG^{R}cT^{R}gG^{R}-NH₂

FluPac-Lys^{L}(BODIPY)-cC^{R}gG^{R}gG^{R}tcgcaG^{R}cT^{R}gG^{R}-NH₂

FluPac-Lys^{L}-G^{R}gC^{R}caaA^{R}cctcgG^{R}cttacC^{R}tG^{R}aA^{R}aT^{R}-NH₂

FluPac-Lys^{L}-G^{R}gC^{R}caaA^{R}cctgcG^{R}cttacC^{R}tG^{R}aA^{R}aT^{R}-NH₂

FluPac-Lys^{L}-A^{R}cC^{R}aT^{R}agcgaG^{R}gT^{R}gA^{R}-NH₂

FluPac-Lys^{L}-G^{R}aC^{R}gA^{R}accatA^{R}gC^{R}gA^{R}-NH₂

FluPac-Lys^{L}-G^{R}aG^{R}gC^{R}agacgA^{R}aC^{R}cA^{R}-NH₂

FluPac-Lys^{L}-A^{R}aG^{R}cA^{R}gccccA^{R}gA^{R}gG^{R}-NH₂

FluPac-Lys^{L}-A^{R}gC^{R}gG^{R}tcageA^{R}aG^{R}cA^{R}-NH₂

FluPac-Lys^{L}-G^{R}aT^{R}gG^{R}acagcG^{R}gT^{R}cA^{R}-NH₂

FluPac-Lys^{L}-C^{R}tA^{R}aA^{R}aacagA^{R}aT^{R}tG^{R}-NH₂

FluPac-Lys^{L}-G^{R}aC^{R}cT^{R}aaaaaC^{R}aG^{R}aA^{R}-NH₂

FluPac-Lys^{L}-G^{R}aT^{R}gG^{R}acctaA^{R}aA^{R}aC^{R}-NH₂

FluPac-Lys^{L}-T^{R}gG^{R}tT^{R}ctggaT^{R}gG^{R}aC^{R}-NH₂

FluPac-Lys^{L}-T^{R}tT^{R}tC^{R}tctgcA^{R}tG^{R}cA^{R}-NH₂

FluPac-Lys^{L}-C^{R}tG^{R}tT^{R}tttctC^{R}tG^{R}cA^{R}-NH₂

FluPac-Lys^{L}-A^{R}gG^{R}tA^{R}ctgttT^{R}tT^{R}cT^{R}-NH₂

FluPac-Lys^{L}-A^{R}tT^{R}gG^{R}agaagA^{R}aG^{R}cC^{R}-NH₂

FluPac-Lys^{L}-T^{R}gA^{R}cA^{R}11111C^{R}gA^{R}aA^{R}-NH₂

FluPac-Lys^{L}-T^{R}gT^{R}cC^{R}aagggT^{R}gA^{R}cA^{R}-NH₂

FluPac-Lys^{L}-C^{R}tT^{R}gT^{R}ccaagG^{R}gT^{R}gA^{R}-NH₂

FluPac-Lys^{L}-A^{R}cC^{R}tT^{R}gtccaA^{R}gG^{R}gT^{R}-NH₂

FluPac-Lys^{L}-A^{R}tA^{R}cC^{R}ttgtcC^{R}aA^{R}gG^{R}-NH₂

FluPac-Lys^{L}-C^{R}tT^{R}aT^{R}acc11G^{R}tC^{R}cA^{R}-NH₂

FluPac-Lys^{L}-A^{R}cT^{R}gC^{R}aacctC^{R}cA^{R}cC^{R}-NH₂

FluPac-Lys^{L}-T^{R}cA^{R}cT^{R}gcaacC^{R}tC^{R}cA^{R}-NH₂

FluPac-Lys^{L}-C^{R}aG^{R}cT^{R}cactgC^{R}aA^{R}cC^{R}-NH₂

FluPac-Lys^{L}-C^{R}tC^{R}aG^{R}ctcacT^{R}gC^{R}aA^{R}-NH₂

FluPac-Lys^{L}-A^{R}tC^{R}tC^{R}agctcA^{R}cT^{R}gC^{R}-NH₂

FluPac-Lys^{L}-C^{R}gA^{R}tC^{R}tcagcT^{R}cA^{R}cT^{R}-NH₂

FluPac-Lys^{L}-C^{R}gC^{R}gA^{R}tctcaG^{R}cT^{R}cA^{R}-NH₂

FluPac-Lys^{L}-G^{R}gC^{R}gC^{R}gatctC^{R}aG^{R}cT^{R}-NH₂

FluPac-Lys^{L}-G^{R}tG^{R}gC^{R}gcgatC^{R}tC^{R}aG^{R}-NH₂

FluPac-Lys^{L}-C^{R}tC^{R}aG^{R}ctcacT^{R}gC^{R}aA^{R}-NH₂

FluPac-Lys^{L}-G^{R}aT^{R}gG^{R}caaacA^{R}gG^{R}aT^{R}-NH₂

FluPac-Lys^{L}-C^{R}aC^{R}cA^{R}agaggA^{R}tG^{R}gC^{R}-NH₂

FluPac-Lys^{L}-A^{R}gA^{R}cC^{R}agcacC^{R}aA^{R}gA^{R}-NH₂

FluPac-Lys^{L}-A^{R}cT^{R}cA^{R}ctgatA^{R}aA^{R}gA^{R}-NH₂

FluPac-Lys^{L}-C^{R}cT^{R}gA^{R}ggactC^{R}aC^{R}tG^{R}-NH₂

FluPac-Lys^{L}-T^{R}cC^{R}cC^{R}acctgA^{R}gG^{R}aC^{R}-NH₂

FluPac-Lys^{L}-T^{R}gG^{R}cC^{R}accttT^{R}tC^{R}tA^{R}-NH₂

FluPac-Lys^{L}-T^{R}tC^{R}tT^{R}ggccaC^{R}cT^{R}tT^{R}-NH₂

FluPac-Lys^{L}-T^{R}tg^{R}GC^{R}ttcttG^{R}gC^{R}cA^{R}-NH₂

FluPac-Lys^{L}-T^{R}tG^{R}gT^{R}tggctT^{R}cT^{R}tG^{R}-NH₂

FluPac-Lys^{L}-T^{R}aC^{R}cT^{R}tattgG^{R}tT^{R}gG^{R}-NH₂

FluPac-Lys^{L}-C^{R}cT^{R}aC^{R}cttatT^{R}gG^{R}tT^{R}-NH₂

FluPac-Lys^{L}-T^{R}gA^{R}cC^{R}tacctT^{R}aT^{R}tG^{R}-NH₂

FluPac-Lys^{L}-G^{R}gG^{R}tG^{R}acctaC^{R}cT^{R}tA^{R}-NH₂

FluPac-Lys^{L}-aG^{R}aG^{R}cagaaC^{R}cT^{R}-NH₂

FluPac-Lys^{L}-aG^{R}aG^{R}cA^{R}gaaccT^{R}tA^{R}c-NH₂

FluPac-Lys^{L}-aG^{R}agC^{R}agaacC^{R}ttaC^{R}t-NH₂

FluPac-Lys^{L}-agagcA^{R}gA^{R}aC^{R}cT^{R}tact-NH₂

FluPac-Lys^{L}-gC^{R}tA^{R}tT^{R}accttA^{R}aC^{R}cC^{R}-NH₂

FluPac-Lys^{L}-cA^{R}aT^{R}cA^{R}gacctA^{R}gG^{R}aA^{R}-NH₂

FluPac-Lys^{L}-tT^{R}cT^{R}gC^{R}tctcgT^{R}cC^{R}tG^{R}-NH₂

FluPac-Lys^{L}-gT^{R}cG^{R}cG^{R}agacaC^{R}gC^{R}tT^{R}-NH₂

FluPac-Lys^{L}-aG^{R}aG^{R}cA^{R}gaaccT^{R}tA^{R}cT^{R}-NH₂

FluPac-Lys^{L}-gT^{R}cG^{R}cT^{R}gtctcC^{R}gC^{R}tT^{R}-NH₂

FluPac-Lys^{L}-cC^{R}tatC^{R}aC^{R}gA^{R}tatgcT^{R}aT^{R}aA^{R}-NH₂

MN-Lys^{L}-tG^{R}cC^{R}tA^{R}ggactC^{R}cA^{R}gC^{R}-NH₂

MN═4-Hydroxy-3-nitro-phenyl acetate-radical

MN-Lys^{L} (TAMRA)-tG^{R}cC^{R}tA^{R}ggactC^{R}cA^{R}gC^{R}-NH₂

MN-Lys^{L} (ATTO)-tG^{R}cC^{R}tA^{R}ggactC^{R}cA^{R}gC^{R}-NH₂

FluPac-Gly-aG^{R}aG^{R}cA^{R}gaaccT^{R}tA^{R}c-NH₂

FluPac-Lys^{D}-cT^{R}gA^{R}aA^{R}ttttcG^{R}aA^{R}gT^{R}-NH₂

FluPac-Lys^{D}-tT^{R}aC^{R}cT^{R}gaaatT^{R}tT^{R}cG^{R}-NH₂

FluPac-Lys^{D}-cG^{R}gC^{R}tT^{R}acctgA^{R}aA^{R}tT^{R}-NH₂

FluPac-Lys^{D}-cT^{R}cG^{R}gC^{R}ttaccT^{R}gA^{R}aA^{R}-NH₂

FluPac-Lys^{D}-aC^{R}cT^{R}cG^{R}gcttaC^{R}cT^{R}gA^{R}-NH₂

FluPac-Lys^{D}-aA^{R}aC^{R}cT^{R}cggctT^{R}aC^{R}cT^{R}-NH₂

FluPac-Lys^{D}-cC^{R}aA^{R}aC^{R}ctcggC^{R}tT^{R}aC^{R}-NH₂

FluPac-Lys^{D}-aA^{R}gG^{R}cC^{R}aaaccT^{R}cG^{R}gC^{R}-NH₂

FluPac-Gly-aG^{R}aG^{R}cA^{R}gaaccT^{R}tA^{R}cT^{R}-NH₂

FluPac-Gly-aG^{R}agcA^{R}gaaccttA^{R}ct-NH₂

FluPac-Gly-aG^{R}aA^{R}gA^{R}cgttcC^{R}aA^{R}cT^{R}-NH₂

FluPac-Gly-aG^{R}cG^{R}aA^{R}gcataT^{R}aT^{R}cC^{R}-NH₂

FluPac-Gly-aC^{R}aG^{R}gA^{R}cgagaG^{R}cA^{R}gA^{R}-NH₂

FluPac-Gly-aC^{R}cT^{R}tA^{R}cttttC^{R}cT^{R}cT^{R}-NH₂

FluPac-Gly-caC^{R}aG^{R}atgacA^{R}tT^{R}aG^{R}-NH₂

FluPac-Gly-cA^{R}aT^{R}cA^{R}gacctA^{R}gG^{R}aA^{R}-NH₂

FluPac-Gly-aC^{R}aC^{R}cC^{R}acaatC^{R}aG^{R}tC^{R}-NH₂

FluPac-Gly-aC^{R}cC^{R}aC^{R}aatcaG^{R}tC^{R}cT^{R}-NH₂

FluPac-Gly-aC^{R}aA^{R}tC^{R}agtccT^{R}aG^{R}aA^{R}-NH₂

FluPac-Gly-aA^{R}tC^{R}aG^{R}tcctaG^{R}aA^{R}aG^{R}-NH₂

FluPac-Gly-tC^{R}aG^{R}tC^{R}ctagaA^{R}aG^{R}aA^{R}-NH₂

FluPac-Gly-aG^{R}tC^{R}cT^{R}agaaaG^{R}aA^{R}aA^{R}-NH₂

FluPac-Gly-gG^{R}aT^{R}gG^{R}actctT^{R}aC^{R}tT^{R}-NH₂

FluPac-Gly-aT^{R}gG^{R}aC^{R}tcttaC^{R}tT^{R}tT^{R}-NH₂

FluPac-Gly-aC^{R}tC^{R}tT^{R}actttT^{R}cA^{R}cC^{R}-NH₂

FluPac-Gly-tC^{R}tT^{R}aC^{R}ttttcA^{R}cC^{R}tG^{R}-NH₂

FluPac-Gly-tT^{R}aC^{R}tT^{R}ttcacC^{R}tG^{R}gG^{R}-NH₂

FluPac-Gly-tT^{R}tT^{R}cA^{R}cctggG^{R}tC^{R}aT^{R}-NH₂

FluPac-Gly-tC^{R}cA^{R}aC^{R}aatcaG^{R}aC^{R}cT^{R}-NH₂

FluPac-Gly-cA^{R}aC^{R}aA^{R}tcagaC^{R}cT^{R}aG^{R}-NH₂

FluPac-Gly-aC^{R}aA^{R}tC^{R}agaccT^{R}aG^{R}gA^{R}-NH₂

FluPac-Gly-aA^{R}tC^{R}aG^{R}acctaG^{R}gA^{R}aA^{R}-NH₂

FluPac-Gly-tC^{R}aG^{R}aC^{R}ctaggA^{R}aA^{R}aC^{R}-NH₂

FluPac-Gly-aG^{R}aC^{R}cT^{R}aggaaA^{R}aC^{R}gG^{R}-NH₂

FluPac-Gly-aC^{R}gA^{R}gA^{R}gcagaA^{R}cC^{R}tT^{R}-NH₂

FluPac-Gly-gA^{R}gA^{R}gC^{R}agaacC^{R}tT^{R}aC^{R}-NH₂

FluPac-Gly-gA^{R}gC^{R}aG^{R}aacctT^{R}aC^{R}tT^{R}-NH₂

FluPac-Gly-gC^{R}aG^{R}aA^{R}ccttaC^{R}tT^{R}tT^{R}-NH₂

FluPac-Gly-aG^{R}aA^{R}cC^{R}ttactT^{R}tT^{R}cC^{R}-NH₂

FluPac-Gly-aA^{R}cC^{R}tT^{R}actttT^{R}cC^{R}tC^{R}-NH₂

FluPac-Gly-cA^{R}gT^{R}cC^{R}tagaaA^{R}gA^{R}aA^{R}-NH₂

FluPac-Gly-aA^{R}aC^{R}cT^{R}cggctT^{R}aC^{R}cT^{R}-NH₂

FluPac-Lys^{D}-cC^{R}gG^{R}gG^{R}tcgcaG^{R}cT^{R}gG^{R}-NH₂

Ac-Lys^{D}-cC^{R}gG^{R}gG^{R}tcgcaG^{R}cT^{R}gG^{R}-NH₂

FluPac-Lys^{D}-cC^{R}ggggtcgC^{R}aG^{R}cT^{R}gG^{R}-NH₂

FluPac-Lys^{D}-aG^{R}aG^{R}cA^{R}gaaccT^{R}tA^{R}cT^{R}-NH₂

Ac-Lys^{D}-aG^{R}aG^{R}cA^{R}gaaccT^{R}tA^{R}cT^{R}-NH₂

FluPac-Lys^{D}-aG^{R}agcagaaC^{R}cT^{R}tA^{R}cT^{R}-NH₂

FluPac-Lys^{D}-cC^{R}aC^{R}aA^{R}tcagtC^{R}cT^{R}aG^{R}-NH₂

Ac-LysD-cC^{R}aC^{R}aA^{R}tcagtC^{R}cT^{R}aG^{R}-NH₂

FluPac-Lys^{D}-cC^{R}acaatcaG^{R}tC^{R}aT^{R}aG^{R}-NH₂

FluPac-Gly-aC^{R}aT^{R}cA^{R}gtctgA^{R}tA^{R}aG^{R}-NH₂

FluPac-Gly-gG^{R}gG^{R}tC^{R}atcaaG^{R}gG^{R}tG^{R}-NH₂

FluPac-Gly-cC^{R}aC^{R}aG^{R}atgacA^{R}tT^{R}aG^{R}-NH₂

FluPac-Gly-C^{R}caC^{R}aA^{R}tcagtC^{R}cT^{R}aG^{R}-NH₂

FluPac-Gly-C^{R}caC^{R}aA^{R}tcagtC^{R}cT^{R}ag-NH₂

FluPac-Gly-ccaC^{R}aA^{R}tcagtC^{R}cT^{R}aG^{R}-NH₂

FluPac-Gly-ccaC^{R}aA^{R}tcagtC^{R}cT^{R}ag-NH₂

FluPac-Gly-cC^{R}aC^{R}aA^{R}tcagtC^{R}cT^{R}aG^{R}-NH₂

FluPac-Lys^{D}(ATTO)-cC^{R}gG^{R}gG^{R}tcgcaG^{R}cT^{R}gG^{R}-NH₂

Ac-Lys^{D}(ATTO)-cC^{R}gG^{R}gG^{R}tcgcaG^{R}cT^{R}gG^{R}-NH₂

FluPac-Lys^{D}(ATTO)-cC^{R}ggggtcgC^{R}aG^{R}cT^{R}gG^{R}-NH₂

FluPac-Lys^{D}(ATTO)-aG^{R}aG^{R}cA^{R}gaaccT^{R}tA^{R}cT^{R}-NH₂

Ac-Lys^{D}(ATTO)-aG^{R}aG^{R}cA^{R}gaaccT^{R}tA^{R}cT^{R}-NH₂

FluPac-Lys^{D}(ATTO)-aG^{R}agcagaaC^{R}cT^{R}tA^{R}cT^{R}-NH₂

FluPac-Lys^{D}(ATTO)-cC^{R}aC^{R}aA^{R}tcagtC^{R}cT^{R}aG^{R}-NH₂

Ac-Lys^{D}(ATTO)-cC^{R}aC^{R}aA^{R}tcagtC^{R}cT^{R}aG^{R}-NH₂

FluPac-Lys^{D}(ATTO)-cC^{R}acaatcaG^{R}tC^{R}cT^{R}aG^{R}-NH₂

FluPac-Lys^{D}(Chol)-gA^{R}gA^{R}gC^{R}agaacC^{R}tT^{R}aC^{R}-NH₂

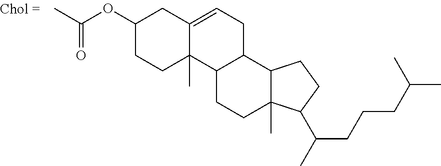 [SMILES: CC(=O)OC1CCC2(C)C(=CCC3C2CCC2(C)C(C(C)CCCC(C)C)CCC32)C1]

FluPac-Lys^{D}(Fol)-gA^{R}gA^{R}gC^{R}agaacCC^{R}tT^{R}aC^{R}-NH₂

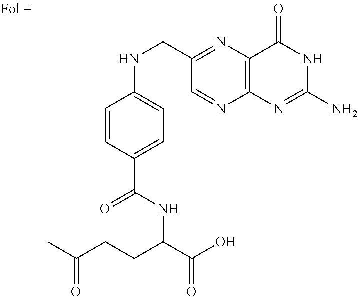 [SMILES: CC(=O)CCC(NC(=O)c1ccc(NCc2cnc3nc(N)[nH]c(=O)c3n2)cc1)C(=O)O]

FluPac-Lys^{D}(Dans)-gA^{R}gA^{R}gC^{R}agaacC^{R}tT^{R}aC^{R}-NH₂

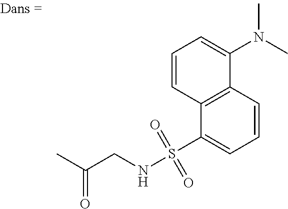 [SMILES: CC(=O)CNS(=O)(=O)c1cccc2c(N(C)C)cccc12]

FluPac-Gly-aG^{R}aG^{R}cA^{R}gaaccT^{R}tA^{R}c-NH₂

FluPac-Gly-aG^{R}aG^{R}Ca^{R}gaaccT^{R}tA^{R}cT^{R}-NH₂

FluPac-Lys^{D}-gA^{R}gC^{R}aG^{R}aacctT^{R}aC^{R}tT^{R}-NH₂

FluPac-Lys^{D}-gA^{R}gaG^{R}cagaaC^{R}ctta^{R}c-NH₂

Ac-Lys^{D}-gA^{R}gaG^{R}cagaaC^{R}ctta^{R}c-NH₂

FluPac-Gly-gA^{R}gcA^{R}gaaccT^{R}tacT^{R}t-NH₂

FluPac-Gly-cagtccT^{R}aG^{R}aA^{R}agaaa-NH₂

FluPac-Gly-gG^{R}ccaA^{R}aC^{R}cT^{R}cggctT^{R}aC^{R}T^{R}-NH₂

FluPac-Gly-cagtccT^{R}aG^{R}aA^{R}agaaa-NH₂

### Example 14

### Improved Bioavailability and Extended Half-Life in Various Organs/Tissues

A ³H labelled N-Phos oligomer and a ³H labelled EP2041161 oligomer are in each case dissolved in PBS (pH 7.1) and administered to mice in a concentration of 10mg/kg by means of an intravenous bolus injection. At various points in time (20 minutes, 1.5 hours, 3 hours, 6 hours, 24 hours, 2 days, 4 days, 8 days and 14 days) blood and 18 different organs/tissues (kidneys, liver, spleen, bone marrow, lymph nodes, lungs, large intestine, small intestine, pancreas, bladder, heart, thymus, stomach, muscle, cerebrum, cerebellum, prostate and skin) were removed from the mice and the ³H-concentration in the respective tissue measured. The pharmacokinetic analysis was performed by means of the validated professional WinNonlin software, Version 4.0.1 (Pharsight Corporation, Mountain View, USA). The radioactivity in the respective organs/tissues (average of three animals per sampling site) was assessed over time without assumed compartments, in order to calculate the bioavailability (expressed as the area under the curve) and the half-lives in the organs/tissues.

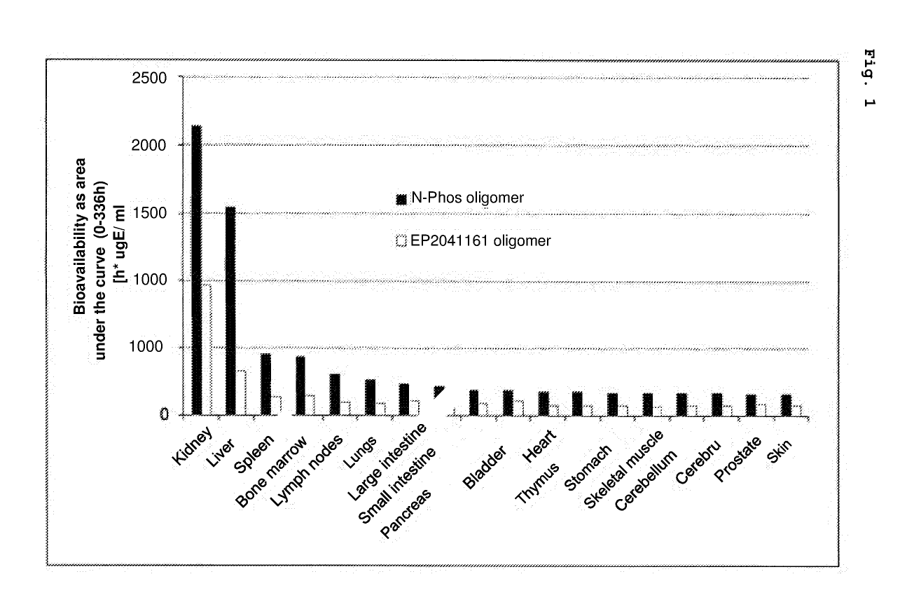

It was found that the bioavailability of the ³H labelled N-Phos oligomer compared to the bioavailability of ³H labelled EP2041161 oligomers over a period of 14 days in all tissues increased by 1.7-4.6 times. The results are shown in FIG. 1.

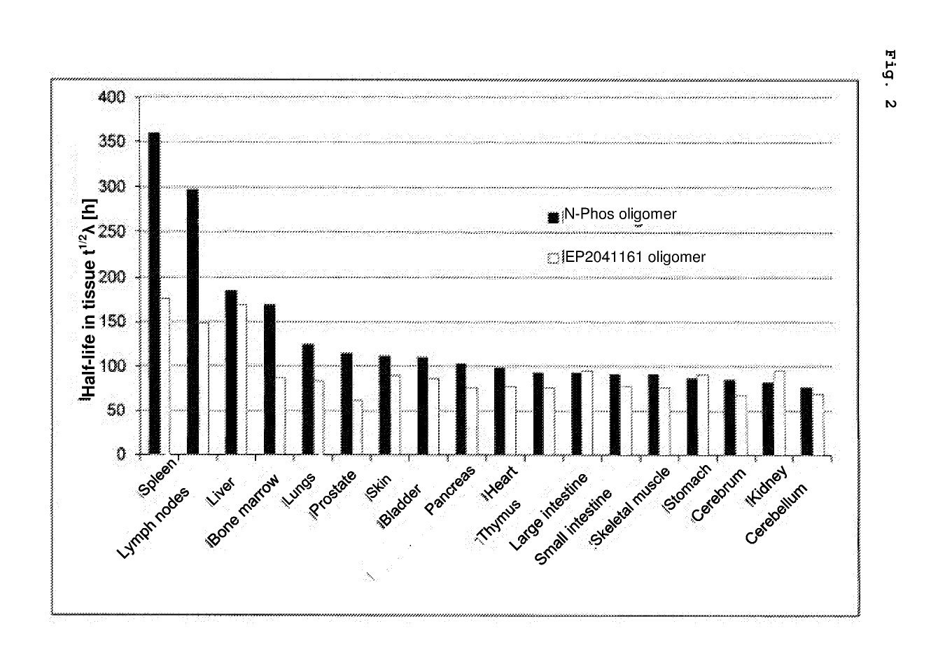

It was also found that the half-life of the ³H labelled N-Phos oligomer compared to the half-life of the ³H labelled EP2041161 oligomer over a period of 14 days is greater in most tissues, in the spleen actually by 2 times. The results are shown in FIG. 2.

### Example 15

### Increased Bonding to Plasma Proteins

The bonding to human serum albumin was determined on a 5 cm HPLC column from Chromtech (4.0×50 mm, 5 μm). Human serum albumin is immobilised on this column, so that over the retention times the binding affinity to human serum albumin can be determined. A 30% isoprop/ammonium acetate buffer (pH 7) was used as the eluent.

The affinity constant is calculated according to the manufacturer using the following formula:

k′=(tr−tm)/tm

tr=retention time of the sample applied

tm=retention time of acetaminophen

With this value the binding (in %) to human serum albumin can be calculated as:

P=100(k′/(k′+1))

A higher value is indicative of an advantageous distribution in vivo. The following table illustrates a significantly stronger binding of the N-Phos oligomer to serum albumin compared to the EP2041161 oligomer.

Table for comparison of the plasma-protein binding
of N-phos oligomers and EP2041161 oligomers

| | Plasma protein | |
|---|---|---|
| | binding (%) | |
| | N-Phos | EP2041161 |
| Sequence: | oligomer^{a} | oligomer^{b} |
| FluPac-Gly-cA^{R}gT^{R}cC^{R}tagaaA^{R}gA^{R}aA^{R}-NH₂ | 94 | 71.5 |

^{a}N-Phos oligomer: R¹ = —CH₂—CH₂—CH₂—CH₂—NH—CH₂—CH₂—P═O(OEt)₂
^{b}EP2041161 oligomer: R¹ = —CH₂—CH₂—CH₂—P═0(OEt)₂

### Example 16

### Improved and Sequence-Independent Water Solubility

Various N-phos oligomers and EP2041161 oligomers are weighed in and as much PBS (pH 7.2) added as theoretically provides a 100 μM solution. Then the OD values of the resulting solutions are measured. Then the OD values of the N-phos oligomers and the EP2041161 oligomers are compared with each other, wherein a higher OD-value corresponds to a higher number of dissolved molecules and thus a higher solubility. Whereas the EP2041161 oligomers, the sequence of which comprises a large number of guanine and cytosine bases, does not allow a 100 μM solution in PBS to be prepared, in which the oligomeric compounds are completely dissolved, when preparing a 100 μM solution the N-phos oligomers are completely dissolved in PBS. The following table illustrates that sequence-independently the water solubility of the N-phos oligomers compared to the EP2041161 oligomers is surprisingly significantly increased.

Table for comparison of the water solubility of N-phos oligomers
and EP2041161

| | N-phos | | EP2041161 | |
|---|---|---|---|---|
| | oligomers^{a} | | oligomers^{b} | |
| | | OD | | OD |
| Sequence | m^{c} | value^{d} | m^{c} | value^{d} |
| FluPac-Lys^{L}-cC^{R}gG^{R}gG^{R}tcgcaG^{R}cT^{R}gG^{R}-NH₂ | 2.6 | 70.4 | 2.4 | 1.4 |
| FluPac-Lys^{L}-cC^{R}gG^{R}gG^{R}tcccgG^{R}gG^{R}gC^{R}-NH₂ | 2.7 | 75.7 | 2.6 | 8.2 |
| FluPac-Lys^{L}-cC^{R}aT^{R}gG^{R}ccgggG^{R}tC^{R}cC^{R}-NH₂ | 2.9 | 81.6 | 2.8 | 2 |
| FluPac-Lys^{L}-gT^{R}tC^{R}gT^{R}ccatgG^{R}cC^{R}gG^{R}-NH₂ | 2.4 | 77.4 | 2.6 | 3 |
| FluPac-Lys^{L}-gG^{R}gG^{R}gA^{R}acagtT^{R}cG^{R}tC^{R}-NH₂ | 2.7 | 72.8 | 2.8 | 8.6 |
| FluPac-Lys^{L}-gA^{R}gG^{R}gG^{R}gaacaG^{R}tT^{R}cG^{R}-NH₂ | 2.7 | 70.6 | 2.4 | 2.1 |
| FluPac-Lys^{L}-cG^{R}gG^{R}aA^{R}gatgaG^{R}gG^{R}gG^{R}-NH₂ | 2.5 | 63.8 | 2.3 | 4.4 |
| FluPac-Lys^{L}-aG^{R}aG^{R}gC^{R}ctgggC^{R}tG^{R}gC^{R}-NH₂ | 2.4 | 86 | 2.4 | 1.8 |
| FluPac-Lys^{L}-cC^{R}aC^{R}aT^{R}aggggC^{R}cA^{R}gA^{R}-NH₂ | 2.6 | 66.9 | 2.5 | 1.3 |
| FluPac-Lys^{L}-tT^{R}gG^{R}gC^{R}tgctcA^{R}aT^{R}gA^{R}-NH₂ | 3 | 77.5 | 2.3 | 11.2 |
| FluPac-Lys^{L}-cG^{R}cG^{R}gA^{R}gcgccC^{R}cT^{R}cG^{R}-NH₂ | 2.9 | 78.8 | 2.8 | 15.8 |
| FluPac-Lys^{L}-tG^{R}gG^{R}gT^{R}gggtcT^{R}tG^{R}gT^{R}-NH₂ | 2.4 | 76.6 | 1 | 5 |

^{a}N-phos oligomers: R¹ = -CH₂-CH₂-CH₂-CH₂-NH-CH₂-CH₂-P = O(OEt)₂
^{b}EP2041161 oligomers: R¹ = -CH₂-CH₂-CH₂-P =O(OEt)₂
^{c}m = Weighed-in quantity [mg]
^{d}OD-value = Measured OD value following preparation of a theoretical 100 μM of solution in PBS;

### Example 17

### Strong Binding to Complementary DNA

An N-Phos oligomer or an EP2041161 oligomer and the sequence complementary DNA oligomer are dissolved in equimolar ratio in magnesium-free and calcium-free physiological PBS buffer. The solution is diluted until in the UV spectrometer an OD value of 0.8 is measured. By means of a heating bath the cuvettes in the UV spectrometer are gradually heated in 1° C. steps from room temperature to 95° C. After each 1° C. step the OD value is determined. The melting point is given by the point of inflection of the resulting curve.

The following table illustrates that the N-Phos oligomer has a higher melting point than the corresponding EP2041161 oligomer. The N-Phos oligomer thus forms a more stable binding to the sequence complementary DNA oligomer.

Table for comparison of the melting point level of N-phos
oligomers and EP2041161 oligomers

| | Melting point (° C.) | |
|---|---|---|
| Sequence: | N-phos | EP2041161 |
| FluPac-Gly-cA^{R}gT^{R}cC^{R}tagaaA^{R}gA^{R}aA^{R}-NH₂ | oligomers^{a} | oligomers^{b} |
| DNA sequence, 100% Match: | 73 | 69 |
| 5′-TTTCTTTCTAGGACTG-3′ | | |

^{a}N-Phos oligomer: R¹ = -CH₂-CH₂-CH₂-CH₂-NH-CH₂-CH₂-P = O(OEt)₂)
^{b}EP2041161 oligomer: R¹ = -CH₂-CH₂-CH₂-P = O(OEt)₂)

### Example 18

### Down-Regulation of NFkB in HeLa-Cells

Two days after cultivation of HeLa cells (source DSMZ), sown in Greiner μClear 384w plates with density of 800 cells/well, N-phos oligomers or EP2041161 oligomers dissolved in PBS are added, so that in each case final concentrations of 0.5, 2.5, and 10 μM in complete medium are obtained. On day 5 after cell sowing 20 μl/well of fresh complete medium are added. On day 7 a change to a starvation medium with 0.1% FCS takes place. On day 8, for stimulation of the cells, 10 ng/ml TNFα (Peprotech) are added to the medium (0.1% FCS, no antibiotics), the cells are fixed after 30 minutes for morphological analysis (4% PFA) and for representation of the most important subcellular structures such as the cell nucleus and cytoplasm, coloured with appropriate dyes and antibodies. Image processing takes place in an ImageXPress Micro automated microscope (MDC). The image analysis is carried out visually with the MetaMorph (MDC) software and then quantitatively with the automated image analysis software Definiens XD (Definiens) using specific algorithms. In this way, by means of the colouring with a Hoechst dye, the number of cell nuclei is determined, which serves as a surrogate for the extent of the cell proliferation and thus for the down-regulation of NFkB by the oligomeric compounds. The following table illustrates on the basis of the lower values of the number of cell nuclei, especially at the concentrations of 2.5 μM and 10 μM, that the effect on the gene expression of the N-phos oligomers on the basis of the increased down-regulation of NFkB is better compared to the EP2041161 oligomers.

```
Table for comparison of the down-regulation of NFkB of N-phos oligomers
  with EP-2041161 oligomers on the basis of the determination of the
  cell proliferation by measuring the number of cell nuclei.
```

| | Number of cell nuclei | | | | | |
|---|---|---|---|---|---|---|
| | (mean normalised) | | | | | |
| | N-Phos | | | EP-2041161 | | |
| | oligomer^{a} | | | oligomer^{b} | | |
| | 0.5 | 2.5 | 10 | 0.5 | 2.5 | 10 |
| Sequence | μM | μM | μM | μM | μM | μM |
| FluPac-Lys^{L}-cC^{R}gG^{R}gG^{R}tcgcaG^{R}cT^{R}gG^{R}- | 97.1 | 92.0 | 68.7 | 99.3 | 102.4 | 111.5 |
| NH₂ | | | | | | |
| FluPac-Lys^{L}-cC^{R}gG^{R}gG^{R}tcccgG^{R}gG^{R}gC^{R}- | 92.1 | 92.7 | 79.7 | 106.2 | 106.7 | 114.5 |
| NH₂ | | | | | | |
| FluPac-Lys^{L}-CC^{R}aT^{R}gG^{R}ccgggG^{R}tC^{R}cC^{R}- | 92.3 | 83.7 | 66.6 | 105.0 | 111.8 | 119.2 |
| NH₂ | | | | | | |

^{a}N-phos oligomers: R¹ = -CH₂-CH₂-CH₂-CH₂-NH-CH₂-CH₂-P = O(OEt)₂
^{b}EP2041161 oligomers: R¹ = -CH₂-CH₂-CH₂-P = O(OEt)₂

### Example 19

### More Powerful Splice Site Modulation of the Target TNFR2 (Exon 7 Skipping) in THPl Cells

The efficacy tests for the oligomeric compounds are performed in THP1 cell culture from ATCC (ATCC TIB-202®). THPl is a human monocyte cell line from an acute monocytic leukaemia patient. THPl cell cultures (in RPMI 1640 medium with 10% FCS) are performed without the use of antibiotics. Mycoplasma tests (with a Venor GeM-Kit from Minerva) are carried out frequently.

On day 1 THP1-cells are placed, with the addition of PMA, at 13,000 cells/well in Greiner collagen I 384W plates (#781956) using a multi-drop dispenser. In this step the cells are treated in complete medium with 10% serum and penicillin/streptomycin. Following sowing, PMA (Sigma #P8139) is added at 100 nM. On day 4, following exchange of the culture medium, the oligomers are added in the concentrations of 0.2, 2 and 20 μM. On day 6 the cells are conditioned by addition of THP1 INFg at 100 U/ml (Peprotech) and also with IFN-γ for 24 h. On day 7, following exchange of the cell culture medium, 5 μg of LPS (Sigma) in starvation medium (0.1% FCS) are added and the culture is stimulated for 24 hours. On day 8 THP1 cells are lysed in lysis buffer Stratec S (#7061311700) and the lysate stored at −80° C. On day 9, using RNA Stratec InviTrap RNA extraction kits, cells (#7061300400) are extracted and stored at −80° C. for further analysis.

The RT reaction is carried out using the LifeTech High Capacity cDNA Kit (#4368813) with RNase inhibitor (#N8080119). The qPCR is carried out with 11 μl reaction volume, using the Bioline SensiMix Sybr qPCR Mastermix (#QT605-20), with specific primers for the mRNAs of the human TNFR2 isoforms, with and without exon 7.

The qPCR reactions are carried out on an ABI PRISM 7900HT system. The RT-qPCR data are checked manually against amplification curves in each well. Relative mRNA of the target gene is normalised to the mRNA quantity of the Rp113a reference gene.

For each concentration of oligomeric compounds tested, the ratio (expressed in percent) of the expression of the induced TNFR2 isoform without exon 7 to the expression of the TFNR2 isoform with exon 7, in each case always relative to the expression of the reference gene Rp113a, is determined. The equipotent effective concentration, EC50, is calculated on the basis of the curve function with the best statistical fit to the individual concentration data (quadratic matching) with the help of the Excel XLfit.5 add-in (IDBS).

The following table illustrates that the N-phos oligomers in the splice site modulation of TNFR2 have a significantly lower EC50 value than the EP2041161 oligomers.

Table for comparison of the effect on the gene expression of N-
Phos oligomers and EP2041161 oligomers in the example of the
splice site modulation of the target TNFR2 in THP1 cells

| | EC50 Wert (μM) | |
|---|---|---|
| | N-Phos | EP2041161- |
| Sequence | oligomers^{a} | oligomers^{b} |
| FluPac-Gly-cA^{R}gT^{R}cC^{R}tagaaA^{R}gA^{R}aA^{R}-NH₂ | 28.0 | 216.6 |
| FluPac-Gly-cA^{R}gT^{R}cC^{R}tagaaA^{R}gA^{R}aa-NH₂ | 84.6 | 274.7 |

^{a}N-phos oligomers: R¹ = -CH₂-CH₂-CH₂-CH₂-NH-CH₂-CH₂-P = O(OEt)₂
^{b}EP2041161 oligomers: R¹ = -CH₂-CH₂-CH₂-P = O(OEt)₂

### Example 20

### Splice Site Modulation of the Target TNFR2 (Exon 7 Skipping) in Mice

On each of days 1, 3 and 5, a treatment group containing 5 mice of the BalB/C (Jackson Labs) strain was injected intravenously with either 50 mg/kg N-Phos oligomer or PBS in the same volume. On day 8 a stimulation of an inflammatory reaction 15 mg/kg LPS (Phenol-LPS of E. coli serotype Ø127: B8, Sigma Cat #L3129 with an endotoxin value of not less than 500,000 EU (endotoxin units)/mg)) is performed. 3 hours after the LPS stimulation the animals are killed and 30 mg each of spleen and mesenteric lymph nodes prepared and immediately frozen in liquid nitrogen. The tissue samples are stored at −80° C. in the freezer until further processing. For the extraction of RNA the tissue fragments of a little under 30 mg (following removal of excess tissue with a scalpel) are immediately transferred to a tube with 300 μl QIAzol reagent and stainless steel beads (Qiagen cat # 69989) for lysis. The extraction of the RNA from the tissue sample is performed with the Qiagen RNeasy 96 Universal Tissue Kit (Qiagen #74881) according to the manufacturer's procedure. The RNA obtained is stored at −80° C. until further use. In the qPCR analysis, for the RT reaction the High Capacity cDNA Reverse Transcription Kit with RNase inhibitor, Invitrogen (cat #4374966) is used according to the manufacturer's procedure. The qPCR reaction mixtures are prepared with the Bioline SensiMix SYBR Mastermix (#QT605-20), 11 ul reaction volume, with SybrGreen-based identification and pre-validated transcript-specific primer pairs in triplicate. The real-time PCR reactions are performed with an ABI PRISM 7900HT system. The RT-qPCR data are manually checked, and the quantities of the target mRNA normalised on the basis of the quantity of mRNA of the RNA reference gene Rp113a. The expression level of the target mRNA, the mRNA isoform of the TNFR2 gene of the mouse without exon 7, was determined as the median of the 5 medians of the measurement in triplicate of the spleen or the mesenteric lymph nodes, normalised to Rp113a in each case for a mouse from the test group of 5 mice.

Table showing the powerful effect on the gene expression of N-Phos
oligomers in the example of the splice site modulation of the
target TNFR2 in mice

| | Median of the expression of | |
|---|---|---|
| | the TNFR2 mRNA isoform | |
| | without exon 7 (expressed as | |
| | a multiple of the RNA | |
| | standard Rpl13a) | |
| | Mesenteric | |
| PBS or N-Phos-Oligomera | lymph nodes | Spleen |
| PBS | 0.000834 | 0.001097 |
| FluPac-Gly-aG_{R}aG_{R}cA_{R}gaaccT_{R}tA_{R}cT_{R}-NH₂ | 0.017613 | 0.027815 |
| FluPac-Gly-gA_{R}gA_{R}cA_{R}agaacC_{R}tT_{R}aC_{R}-NH₂ | 0.038645 | 0.032936 |

^{a}N-phos oligomers: R¹ = -CH₂-CH₂-CH₂-CH₂-NH-CH₂-CH₂-P = O(OEt)₂

### Example 21

### Efficacy Comparison Between an N-Phos Oligomer According to the Invention, an EP 2041161 Oligomer and a U.S. Pat. No. 5,719,262 Oligomer in the Splice Site Modulation of the Target TNFR2 (Exon 7 Skipping) in THP1 Cells

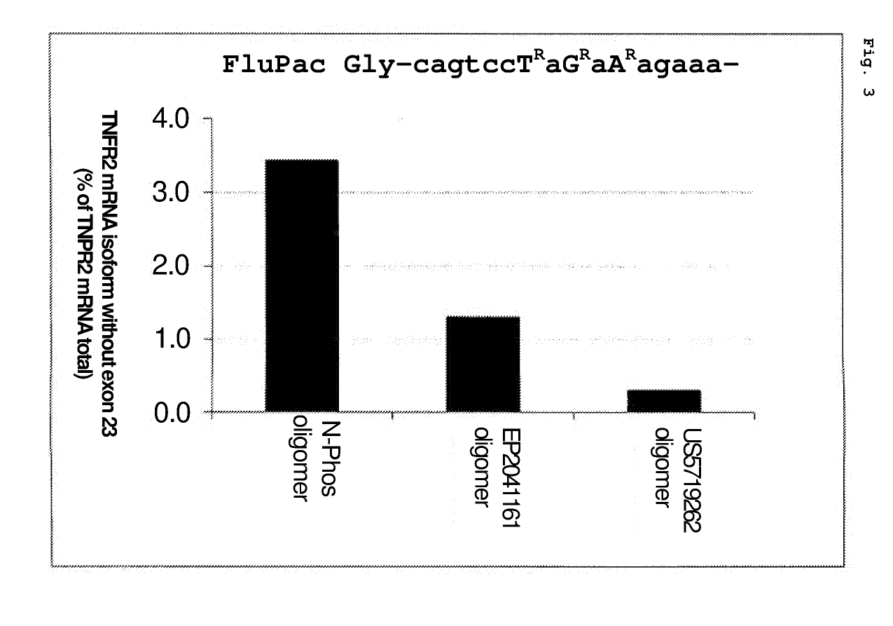

The experiment was performed as described in Example 19. The results are shown in the following table and FIG. 3.

The N-Phos oligomer according to invention, compared to the EP 2041161 oligomer, demonstrates a 2.6 times more powerful effect in the splice site modulation of the target TNFR2 in THP1 cells, whereas the modulation of the target TNFR2 in THP1 cells by the U.S. Pat. No. 5,719,262 oligomer is virtually zero.

Table for comparison of the effect on the gene expression of N-Phos
oligomers and EP2041161 oligomers and US5719262 oligomers in the
example of the splice site modulation of the target TNFR_2 in THP1
cells in a concentration of 10 μM.

| | Ratio of TNFR2 mRNA isoform | | |
|---|---|---|---|
| | without exon 7 to TNFR2 mRNA | | |
| | total in % | | |
| | N-Phos | EP 2041161 | US5719262 |
| Sequence | oligomer^{a} | oligomer^{b} | oligomer^{c} |
| FluPac-Gly-cagtccT^{R}aG^{R}aA^{R}agaaa-NH² | 3.4 | 1.3 | 0.3 |

^{a}N-phos oligomers: R¹ = -CH₂-CH₂-CH₂-CH₂-NH-CH₂-CH₂-P = O(OEt)₂
^{b}EP2041161 oligomers: R¹ = -CH₂-CH₂-CH₂-P = O(OEt)₂
^{c}US5719262-O1igomer: R¹ = -CH₂-CH₂-CH₂-CH₂-NH₂

### Example 22

### Effect of N-Phos Oligomers According to the Invention with Differing Radicals U on the Splice Site Modulation of the Target TNFR2 (Exon 7 Skipping) in the Lungs of Mice

The experiment was performed as described in Example 20.

The tested N-phos oligomers according to the invention of formula (VI) with a radical U according to the general formula VII and a group of the formula IXc (cholesterol derivative) or IXd (folic acid derivative) as R⁴⁶ demonstrate a powerful effect on the gene expression in the splice site modulation of the target TNFR2.

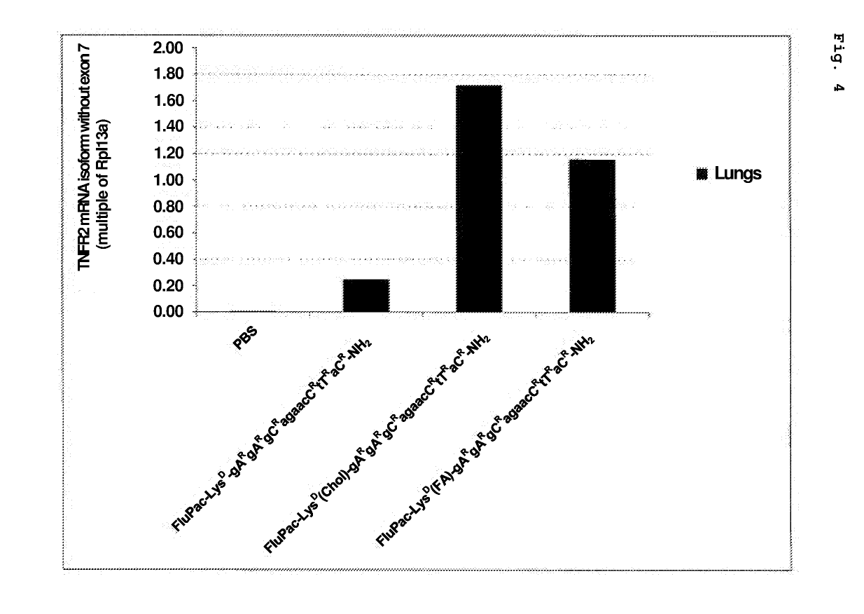

By way of example, the effect in the lungs when the cholesterol derivative is used is 560 times greater and with the folic acid derivative 378 times greater compared to the PBS negative control. The results are shown in FIG. 4.

### Example 23

### Effect of N-Phos Oligomers According to the Invention with Different Nucleobase Sequence, Radicals U, and Differences in the Number and Position of the Groups of the General Formulae (IV) and (V) According to the General Formula (VI) on the Splice Site Modulation of the Target TNFR2 (Exon 7 Skipping) in the Kidneys, Liver and Lungs of Mice

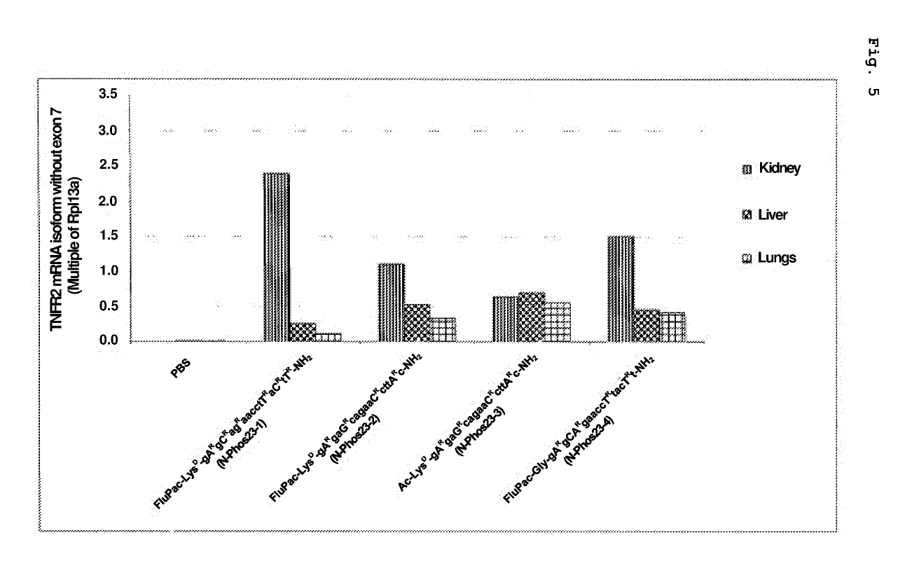

The experiment was performed with the N-phos oligomers according to the invention N-Phos 23-1, N-Phos 23-2, N-Phos-23-4 (see FIG. 5). The experiment was performed as described in Example 20.

All N-phos oligomers according to the invention tested demonstrate in various mouse tissues (kidneys, liver and lung) very powerful effects on the gene expression of the mRNA isoform without exon 7. In the kidneys, for example, the effect of N-Phos 23-1 is 1,983 times greater than the PBS negative control. The results are shown in FIG. 5.

### Example 24

### Efficacy Comparison Between N-Phos Oligomers According to the Invention and EP 2041161 Oligomers in the Splice Site Modulation of the Target TNFR2 (Exon 7 Skipping) in the Kidneys of Mice

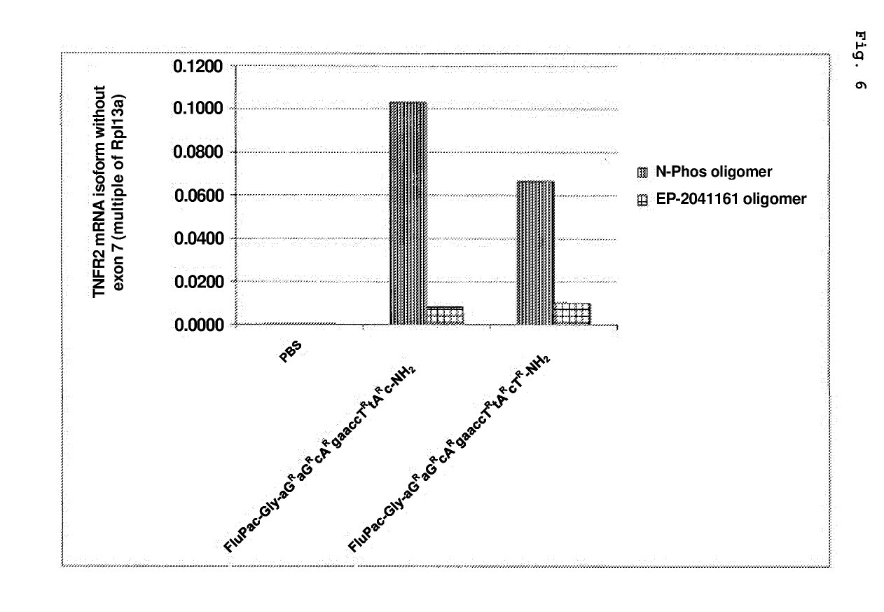

2 variants of N-phos oligomers (variant 1: sum of all repeat units Yd, Zf, Yg, and Zj according to the general formula (VI)=15 or 14; Variant 2: number and position of the groups of the general formula (IV and (V) according to the general formula (VI)) and the corresponding variants of EP2041161 oligomers were tested. The variants tested are shown in FIG. 6. The experiment was performed as described in Example 20, with the difference that in this experiment the animals had already been killed 2 hours after LPS stimulation.

The effect of the N-phos oligomers on the gene expression of the mRNA isoform without exon 7 is, in direct comparison with the EP2041161 oligomers, 12.6 or 6.7 times more powerful. The results are shown in FIG. 6.

### Example 25

### In Vivo Effect of an N-Phos Oligomer According to the Invention with 20 Building Blocks on the Splice Site Modulation of the Target Dystrophin (Exon 23 Skipping) in the Muscle of Mdx Mice

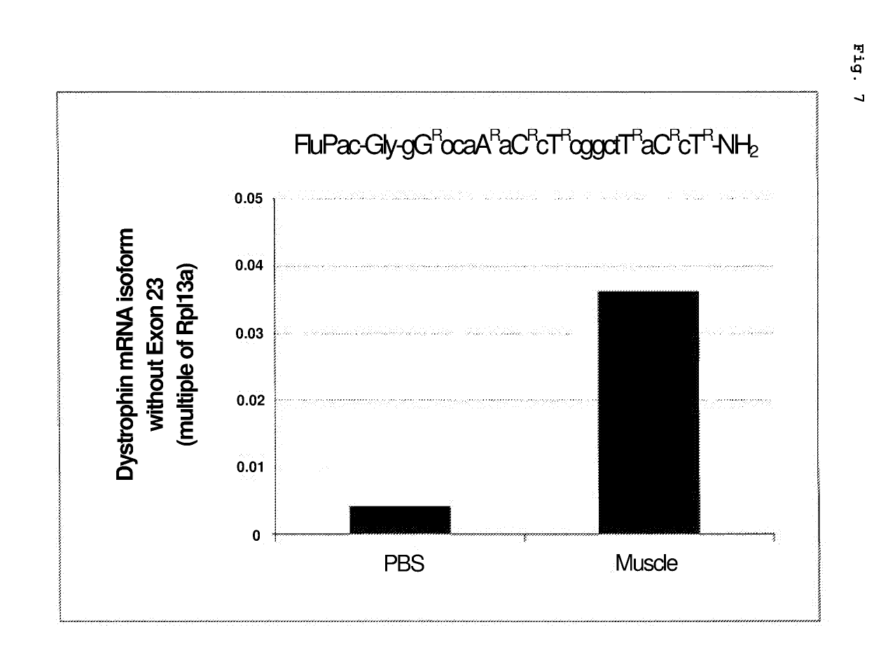

The experiment was performed with an N-Phos oligomer according to the invention with 20 building blocks (sum of all repeat units Yd, Zf, Yg, and Zj according to the general formula (VI)=19). The tested compound is shown in FIG. 7. This experiment was performed as described in Example 20, with the difference that in this experiment mice of the C57BL/10ScSn-Dmdmdx/J (Jackson Labs) strain were used, the animals were not stimulated with LPS, and they were killed on day 15. The expression level of the target mRNA, of the mRNA isoform of the dystrophin gene of the mouse without the exon 23, was determined as the median of the 5 medians of the measurement in triplicate of the muscle, normalised to Rp113a.

The N-Phos oligomer according to the invention with 20 building blocks demonstrates in the muscle a 9 times more powerful effect in vivo on the gene expression of the mRNA isoform without exon 23 compared to the PBS control group. The result is shown in FIG. 7.

1. Compound of general formula (I): 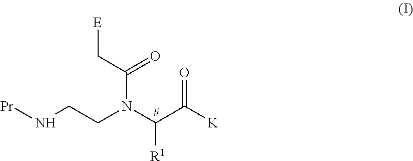
wherein
K represents a carboxylic acid active ester group or —O—R_{M}; wherein R_{M} represents an H atom, a methyl, ethyl, allyl, benzyl, phenyl, tert.-butyl, or a trimethylsilyl group;
Pr represents an H atom or an amino protective group;
# denotes an asymmetric C atom;
E represents an adeninyl, cytosinyl, pseudoisocytosinyl, guaninyl, thyminyl, uracilyl or phenyl group substituted as necessary with a nucleobase protective group;
R¹ is a group, represented by the general formula (II): 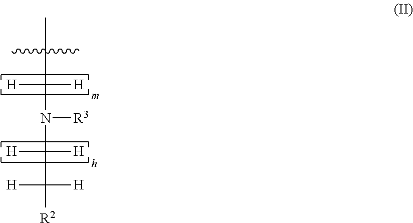
wherein
R² is a phosphonic acid ester group or a phosphonic acid group;
R³ is an H atom, or an amino protective group;
m is 1, 2, 3 or 4; and
h is 0, 1,2or 3;
provided that for the sum of m and h in the general formula (II): 2≦x≦5.

2. Compound according to claim 1, wherein E represents a thyminyl, uracilyl, phenyl, N2-acetyl-guaninyl, N2-isobutyryl-guaninyl, N2-benzyloxycarbonyl-guaninyl, N2-(4-methoxyphenyl)-diphenylmethyl-guaninyl, N2-benzhydryloxycarbonyl-guaninyl, N2-di-benzhydryloxycarbonyl-guaninyl, N2-tert-butyloxycarbonyl-guaninyl, N2-di-tert-butyloxycarbonyl-guaninyl, N6-benzyloxycarbonyl-adeninyl, N6-(4-methoxyphenyl)-diphenylmethyl-adeninyl, N6-anisoyl-adeninyl, N6-benzhydryloxycarbonyl-adeninyl, N6-di-benzhydryloxycarbonyl-adeninyl, N6-tert-butyloxycarbonyl-adeninyl, N6-di-tert-butyloxycarbonyl-adeninyl, O6-benzylguaninyl, N2-acetyl-O6-diphenylcar-bamoyl-guaninyl, N2-isobutyryl-O6-diphenylcarbamoyl-guaninyl, N2-benzyloxycarbonyl-O6-diphenylcarbamoyl-guaninyl, N2-(4-methoxyphenyl)-diphenylmethyl-O6-diphenylcarbamoyl-guaninyl, N2-benzhydryloxycarbonyl-O6-diphenylcarbamoyl-guaninyl, N4-benzyloxycarbonyl-cytosinyl, N4-(4-methoxyphenyl)-diphenyl-methyl-cytosinyl, N4-4-tert.butylbenzoyl-cytosinyl, N4-benz-hydryloxycarbonyl-cytosinyl, N4-di-benzhydryloxycarbonyl-cytosinyl, N4-tert-butyloxycarbonyl-cytosinyl, N4-di-tert-butyloxycarbonyl-cytosinyl, N2-benzyloxycarbonyl-pseudo-isocytosinyl, N2-(4-methoxyphenyl)-diphenylmethyl-pseudoisocyto-sinyl, N2-4-tert.-butylbenzoyl-pseudoisocytosinyl, N2-benz-hydryloxycarbonyl-pseudoisocytosinyl, N2-di-benzhydryloxycarbonyl pseudoisocytosinyl, N2-tert-butyloxycarbonyl-pseudoisocytosinyl or an N2-di-tert-butyloxycarbonyl-pseudoisocytosinyl group.

3. Compound according to claim 2, wherein E represents a thyminyl, uracilyl, phenyl, N2-benzyloxycarbonyl-guaninyl, N2-benzhydryloxycarbonyl-guaninyl, N2-tert-butyloxycarbonyl-guaninyl, N2-benzyloxycarbonyl-O6-diphenylcarbamoyl-guaninyl, N2-benzhydryloxycarbonyl-O6-diphenylcarbamoyl-guaninyl, N6-benzyloxycarbonyl-adeninyl, N6-benzhydryloxycarbonyl-adeninyl, N6-tert-butyloxycarbonyl-adeninyl, N6-di-tert-butyloxycarbonyl-adeninyl, N4-benzyloxycarbonyl-cytosinyl, N4-benz-hydryloxycarbonyl-cytosinyl, N4-di-tert-butyloxycarbonyl-cytosinyl, N2-benzyloxycarbonyl-pseudo-isocytosinyl, N2-benz-hydryloxycarbonyl-pseudoisocytosinyl or an N2-tert-butyloxycarbonyl-pseudoisocytosinyl group.

4. Compound according to claim 1, wherein R² represents a phosphonic acid ester group of the formula —P(═O)(OV)₂ or —P(═O)(OV)(OH); and each V independently represents a methyl, ethyl, cyclohexyl, or benzyl group.

5. Compound according to claim 1, wherein R³ is an H atom.

6. Compound according to claim 1, wherein R³ represents an oxocarbamate, thiocarbamate, or an Mmt protective group.

7. Compound, represented by the general formula (VI): 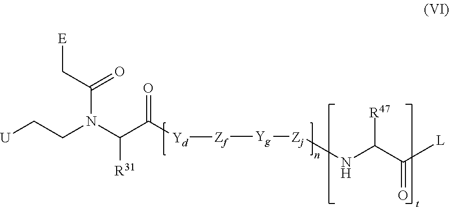
wherein
each Y in each case independently represents a group of general formula (IV): 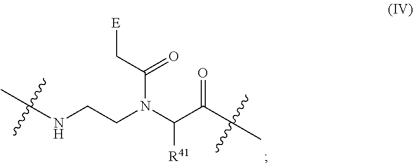
each Z in each case independently represents a group of general formula (V): 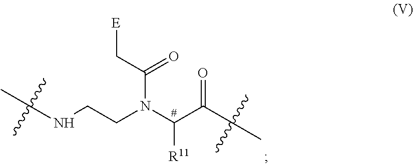
wherein
each E in each case independently represents an adeninyl, cytosinyl, pseudoisocytosinyl, guaninyl, thyminyl, uracilyl, or phenyl group;
# denotes an asymmetric C atom;
each R⁴¹ in each case represents an H atom;
each R¹¹ represents a group —(CH₂)ₘ—NH—(CH₂)ₕ—CH₂—R¹²;
wherein R¹² in each case independently represents a phosphonic acid ester group of the formula —P(═O)(OV)₂ or —P(═O)(OV)(OH); and each V in each case independently represents a methyl, ethyl, cyclohexyl, or benzyl group;
m in each case independently is 1, 2, 3 or 4 and h in each case independently is 0, 1, 2, or 3; provided that for the sum of m and h: 2≦x≦5;
d in each case independently is 0, 1, 2, 3 or 4;
f in each case independently is 0, 1, 2, 3 or 4;
g in each case independently is 0, 1, 2, 3 or 4;
j in each case independently is 0, 1, 2, 3 or 4;
n=0, 1, 2, 3, 4, 5, 6, 7 or 8;
provided that for the sum of all repeat units Yd, Zf, Yg, and Zj in the general formula (VI): 7≦x≦30; and at least one of the variables f or j represents an integer from 1 to 5;
provided that for the ratio (sum of repeat units Zf and Zj): (sum of all repeat units Yd, Zf, Yg, and Zj) in the general formula (VI): 0.1≦x≦0.5;
R³¹ represents an H atom; a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; or a group —(CH₂)ₘ—NH—(CH₂)ₕ—CH₂—R¹²; wherein R¹² is a phosphonic acid ester group or a phosphonic acid group; m represents an integer from 1 to 5; and h represents an integer from 0 to 4; provided that for the sum of m and h: 2≦x≦5;
R⁴⁷ in each case independently is an H atom, or a side chain of the amino acid lysine, ornithine, or arginine;
t=0, 1, 2, 3, 4, 5, 6, 7 or 8;
L represents OH, OEt, NH₂ or —NHNH₂;
U represents a group of general formula (VII): 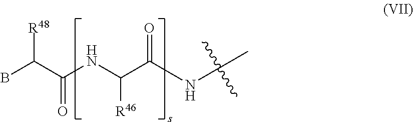
wherein B represents an H atom, a phenyl group, or a substituted phenyl group, substituted with 1 to 3 substituents, selected from the group, consisting of OH, F, Cl, Br, I and NO₂; R⁴⁸ is an H atom; and
(i) each R⁴⁶ in each case independently of each other represents an H atom, or a side chain of the amino acid alanine, arginine, asparagine, aspartic acid, cysteine, glutamine, glutamic acid, histidine, isoleucine, leucine, lysine, methionine, ornithine, phenylalanine, proline, histidine, serine, threonine, tryptophan, tyrosine, or valine; and s is 0, 1, 2, 3, 4, 5, 6, 7 or 8; or
(ii) each R⁴⁶ in each case independently of each other represents an H atom, or a group of the formula (IXb): 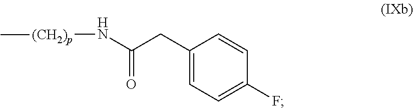
a group of the formula (IXc): 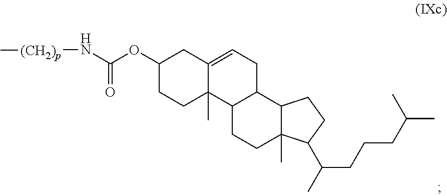
a group of the formula (IXd): 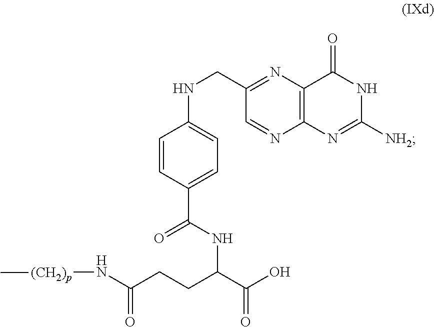
or a group of the formula (IXe): 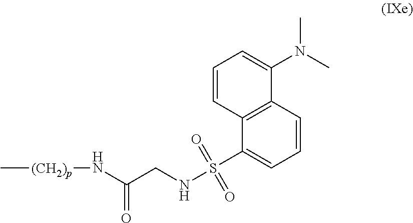
p in formulae (IXb), (IXc), (IXd), and (IXe) represents the number 3 or 4; and s=1, 2, 3 or 4.

8. Compound according to claim 7, wherein each R¹¹ in each case represents a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

9. Compound according to claim wherein each R³¹ represents an H atom, a side chain of the amino acid lysine, ornithine, arginine, histidine, tryptophan, tyrosine, threonine or serine, a group of the formula —CH₂—CH₂—CH₂—CH₂—NH—CH₂—CH₂—P═O(OEt)₂, or a group of the formula —CH₂—CH₂—CH₂—NH—CH₂—CH₂—P═O(OEt)₂.

10. (canceled)

11. Pharmaceutical composition containing at least one compound according to claim 7, and optionally at least one carrier and/or at least one adjuvant.

12. A method for treating a subject suffering or susceptible to a disease, comprising administering to the subject an effective amount of a compound of claim 7.

13. The method of claim 12 wherein the subject is suffering from a viral disease, cancer, a neuromuscular disease, an inflammatory disease, an autoimmune disease, a neurological disease, or a metabolic condition.

14. The method of claim 12 wherein the subject is suffering from human immunodeficiency virus (HIV), hepatitis B virus and hepatitis C virus, human papilloma virus (HPV), skin cancer, lung cancer, liver cancer, prostate cancer, leukaemia, brain tumours, muscular dystrophy, spinal muscular atrophy, asthma, rheumatoid arthritis, psoriasis, Crohn's disease, multiple sclerosis, Parkinson's disease, high cholesterol or obesity.


SEQUENCE LISTING (1 sequence)

SEQ ID NO: 1 - DNA, 16 bp, Artificial Sequence
  DNA Oligomer for thermal stability (Tm) measurements of PNAs
tttctttctaggactg